(19) United States
(12) Patent Application Publication
(10) Pub. No.: US 2025/0177674 A1
(43) Pub. Date: Jun. 5, 2025

(54) EMERGENCY VENTILATING AND MONITORING SYSTEM AND METHODS

(71) Applicant: Alexander Polyansky, Mount Sinai, NY (US)

(72) Inventor: Alexander Polyansky, Mount Sinai, NY (US)

(21) Appl. No.: 18/964,415

(22) Filed: Nov. 30, 2024

Related U.S. Application Data
(60) Provisional application No. 63/604,865, filed on Nov. 30, 2023.

(51) Int. Cl. A61M 16/06; A61M 16/00; A61M 16/10; A61M 16/20

(52) U.S. Cl. CPC A61M 16/0616; A61M 16/0066; A61M 16/022; A61M 16/1005; A61M 16/208; A61M 2205/502

(57) ABSTRACT

An emergency ventilating and monitoring system comprising the emergency ventilating and monitoring apparatus (EVMA), the apparatus comprising: a mask member comprising a vitals sensor system, a gas delivery assembly, a controller, a user interface, a one-way inspiration valve, a one-way expiration valve, and at least one system monitoring means, and configured to be connected to a source of power. The system may include axillary and supporting modules. The system is configured to operate in a feedback control mode and may be configured to operate in CPR and non-rebreather sub-modes. The system may be configured to receive an external oxygen source and at least one of the auxiliary modules to the mask member.

This is a non-provisional utility patent application filed under 35 U.S.C. 111(a)

### CROSS-REFERENCE TO RELATED APPLICATIONS

This application claims the benefit of U.S. Provisional Application No. 63/604,865 entitled “EMERGENCY VENTILATING AND MONITORING APPARATUS AND METHODS” filed on Nov. 30, 2023, the disclosure of which is incorporated herein by reference in its entirety.

### FIELD OF INVENTION

The present invention relates to critical care medical devices, and more specifically to an automated compact and portable ventilating and monitoring apparatus and system that may be used in emergency and critical care setting and configured to monitor physiological parameters and administer ventilation to individuals. The present invention further relates to methods of using the apparatus and the system.

### BACKGROUND

Different types of ventilation devices currently exist. One of the current solutions includes bag valve masks (BVMs) and automatic ventilators equipped with a mask configured to deliver gas flow to a patient. However, these devices have various disadvantages. For instance, the BVM often requires a person to manually operate it and most automatic ventilators are bulky and designed for use in a hospital setting. Moreover, the physiological parameters of a patient are still routinely accessed by multitude of separate devices that are applied to various areas of patient's body; and if such parameters are being measured by a single care provider, that requires the provider to sequentially use such devices by switching between one device to another, thus increasing time required for full patient assessment that in emergency setting may be detrimental. In situations wherein multiple care providers are available for assessment, and several devices may be applied simultaneously, other problems arise; and though, time required for assessment may be decreased, the chances that some of the parameters may be miscommunicated between providers or not become available at the same time and may greatly increase probability of medical error and/or substandard patient care.

Wherein, some automatic ventilators have capability to measure some physiological parameters, such capability is normally delivered by multiple auxiliary devices connected to such ventilators, and wherein the devices are applied to various areas of patient's body. Moreover, existing devices are often bulky, operable to measure only very few parameters, thus provide limited picture of the current condition of a patient and may further require multiple providers to properly apply the auxiliary devices to different areas of patient body in a timely manner. Even lighter, somewhat portable solutions that currently exist are not free of aforementioned disadvantages.

In addition, in emergency settings multiple care providers may not be always present, while timely and extensive assessment, monitoring and treatment is often critical and may be lifesaving. Thus, a device/system that would be substantially free of mentioned drawbacks is highly desirable.

Applicant has recognized that here is a prominent need in a device and/or system that would be operable not only to administer ventilation to a patient, but also to provide immediate assessment and monitoring of the patient, wherein such device would be configured as a portable and compact apparatus that may only require a single care provider to efficiently operate. Such apparatus, preferably, would be configured to measure a multitude of physiological parameters thus reducing the need for multitude of auxiliary devices or attachments.

This background information is provided to reveal information believed by the applicant to be of possible relevance to the present invention. No admission is necessarily intended, nor should it be construed that any of the preceding information constitutes prior art against the present disclosure.

### SUMMARY OF THE INVENTION

In view of the foregoing and other problems, embodiments of the present invention advantageously fill industry needs by providing a one-person-operable, compact and portable, all-in-one ventilating and monitoring system requiring only a single area of body contact. The system configured to provide critical assessment, monitoring and ventilating a patient by measuring a multitude of physiological parameters and administering appropriate ventilation to the patient based at least partially on one or more of the measured parameters, wherein the physiological parameters being measured by the vitals sensor system integrated in the mask member. The system is configured to operate in a feedback-controlled mode and may be used for automated or at least partially attended monitoring and ventilation of the patient. Moreover, the system may be configured to operate in different ventilation sub-modes and switch between the sub-modes according to an imminent need.

These and other objects, features, and advantages according to embodiments of present invention may be provided by an emergency ventilating and monitoring system comprising an emergency ventilating and monitoring apparatus (EVMA).

The invention disclosed herein is an elegant and robust and time-saving solution that substantially eliminates the need for critical care providers to be engaged in measuring, evaluating and tracking the multitude of physiological parameters of the patient by applying multiple devices to multiple body areas, while may as well providing needed ventilation. Embodiments of the present invention offer an unsurpassed convenience, particularly to first responders, such as EMTs and paramedics, by enabling a single care provider to accomplish most of the initial assessment, ventilating, and monitoring tasks in a fast, efficient, accurate and substantially hands-free manner, and thus allowing the critical care provider to focus on and administering additional lifesaving treatments. Moreover, the invention greatly improves quality of care by dramatically reducing initial assessment time and providing a fuller clinical picture by simultaneously measuring and making available to the care provider the multitude of physiological parameters of the patient.

Though the system, and its components may be particularly advantageous in critical and emergency care settings, it may be used in stationary setting such hospitals and long-term care facilities. Although the system is primarily envisioned to be used by a medical care providers, it is also contemplated that the system may be administered by medically untrained individuals or self-administered by a patient when practical.

The embodiments of the invention further relate to methods of using the system comprising EVMA.

The disclosure further relates to the system comprising auxiliary modules that may be configured to advantageously couple to the EVMA for delivering an optimal patient care. Moreover, the disclosure relates to unique components of the EVMA that may be adaptable to being advantageously used outside the exemplary system and EVMA configurations disclosed herein as components for other devices or solo.

These and other objects, features, and advantages according to embodiments of present invention may be provided by the emergency ventilating and monitoring system comprising the emergency ventilating and monitoring apparatus (EVMA), the apparatus comprising: a mask member comprising a vitals sensor system, a gas delivery assembly, a controller, a user interface, a one-way inspiration valve, a one-way expiration valve, and at least one system monitoring means. The system is configured to operate in a feedback control mode and configured to be connected to a source of power. In some embodiments the system may further include at least one axillary module. In some embodiments the system may be configured to operate in at least one of CPR and non-rebreather sub-modes. In some embodiments the system may be configured to receive an external oxygen source to the mask member. In some embodiments, the system may be configured to receive at least one of the auxiliary modules to the mask member. The system may further include at least one of the supporting modules.

The mask member includes a mask body and a vitals sensor system. The mask body has an inner surface configured for engagement with at least facial area of the subject, and an opposed outer surface. The mask body includes an inlet portion and an outlet portion; the inlet portion comprising an inlet opening defined by the mask body and extending between the inner and the outer surfaces of the mask body, and the outlet portion comprising an outlet opening defined by the mask body and extending between the inner and the outer surfaces of the mask body. The vitals sensor system (VSS) operatively associated and/or connected to the mask member and configured to measure data/signals associated with a plurality of physiological parameters of a patient.

The vitals sensor system of the mask member includes one or more sensors configured for measuring at least one, but preferably multiple physiological parameters of a patient and making the data available to a care provider. The vitals sensor system (VSS) may include, but not limited to the sensors/devices configured to measure temperature, blood pressure, blood glucose level, heart rate, pulse, oxygen blood saturation (SpO2); partial pressure of carbon dioxide CO₂ in the exhaled breath, positive end-expiratory pressure (PEEP), tidal volume, cardiac output, and any combinations thereof. The sensors of the vitals sensor system may be imbedded between the inner and outer surfaces of the mask body, may be connected to the mask body and/or mask body components, and/or connected to the inner and/or outer surface of the mask body and/or its components, may be positioned within the inlet and/or outlet portions of the mask member, for instance within inlet and/or outlet openings of the mask member, or be located elsewhere in the mask member; for instance, at least some of the sensors may be positioned on/in fastening means and/or chin support. For instance, at least some of the sensors may be connected to the external surface of the mask body or to other components connected to the external surface of the mask body and placed operatively connected to the interior of the mask. internal surfaces of the mask body/its components via electrical wires, tubes or other conduits. In some embodiments, there may be multiple sensors configured to measure the same physiological parameter, wherein the controller is operable to average the data received from such sensors.

In some embodiments the mask member may include configured to secure the mask body on the patient's head. In some embodiments, fastening means the mask member may further include a chin support. While in some embodiments, all sensors of the vitals sensor system may be integrated in the mask member, for instance in the mask body, in some alternative embodiments at least one of the sensors may be connected to or integrated within the fastening means and/or chin support. Moreover, on some embodiments all sensors of the VSS may be integrated in/connected to the fastening means. For instance, one or more of occipital, posterior auricular, superficial temporal, facial, mental and submental and carotid arteries locations on the subject/patient head may be utilized to measure at least one of the following physiological parameters: temperature, blood pressure, blood glucose level, heart rate, oxygen blood saturation (SpO2); and wherein at least one sensor of the VSS configured for measuring of at least one of the aforementioned parameters may be connected to/imbedded in the fastening means and/or chin support and positioned to be in contact with at least one of the aforementioned arteries when the mask member is fastened on to the subject's head.

The gas delivery assembly positioned operatively connected to the inlet opening of the mask body and configured to generate and direct an output of a gas flow to the inlet portion of the mask body. The gas delivery assembly may be positioned in direct or indirect operative communication or connection with the inlet opening. For example, and without limitation, the gas delivery assembly may be connected to a structure included in an inlet portion of the mask body; for instance, to an intake hatch. Moreover, in some embodiments, at least one of the auxiliary modules may be positioned between the mask member and the gas delivery assembly and operable to deliver treatment into a gas flow directed to the inlet opening. The gas delivery assembly is operable to deliver the gas flow to the mask body and may include but not limited to at least one of the following gas delivery units: a pump, a blower, a compressible bag, a self-inflating bag, a pressurized vessel and combinations thereof, or other devices known in the art that are operable to deliver directed intermittent and/or continuous gaseous output. The gas delivery unit may include an inlet port and an outlet port, wherein the inlet port configured for a gas intake and the outlet port configured for gas output. In some embodiments, the gas delivery assembly may include a pump member. The pump member may include a gas conduit positioned between the pump member and the mask member and configured to direct gas flow from the pump member to the mask member. The gas conduit may include the first end and opposing second end, wherein the first end is configured to couple to the inlet portion of the mask member and the second end is configured to couple to the pump member.

The controller operatively connected to at least the gas delivery assembly, the vitals sensor system of the mask member, and the user interface through direct electrical connection and/or wirelessly. The controller may include at least one of: a logic circuit, a computer, a processor, the internal and external memory/data storage for data recording and storage of instructions/algorithms for logic circuits, storage of reference data and received measured data for at least one parameter measured by the sensor system and/or system control means, wired and/or wireless communication modules; and any combinations thereof. The controller is configured to receive the input from a user (such as health care provider) through the user interphase. The controller is configured to receive physiological parameters data from the vitals sensor system of the mask member; process the data according to pre-programmed algorithms and provide autonomous control of the gas delivery assembly output ventilation parameters and/or auxiliary units, supporting modules parameters based at least partially on data associated with at least one, but preferably multiple, physiological parameters received from the vitals sensor system, and/or an input (or the absence thereof) received from the user. The system is operable in a feedback-controlled mode, wherein the controller is configured to control and constantly adjust the output ventilation parameters of the gas delivery assembly (for example, and without limitation, tidal volume, respiratory rate (breaths per a time period), positive end-expiratory pressure (PEEP), fraction of inspired oxygen (FiO2), minute ventilation, airway pressure, mode of ventilation, a volume of gas administered by the gas delivery assembly per a set time period/pump cycle, a rate of gas delivery by the gas delivery assembly and any combinations thereof) based at least partially on the data associated with at least two of physiological parameters most recently received from the vitals sensor system, and a most recent input received or the absence thereof from the user. In some embodiments, the controller is operable to control and constantly adjust the output ventilation parameters based at least partially on the data measured/associated with at least heart rate and oxygen saturation received form the vitals sensor system of the mask member, but not limited thereto; while in other embodiments, based at least partially on the data measured/associated with at least heart rate and oxygen saturation and blood pressure received form the vitals sensor system of the mask member, but not limited thereto. However, in some embodiments, at least one of the other physiological parameters measured by the VSS one or more additional physiological parameters measured by the VSS may be used by the controller to control and constantly adjust the output ventilation parameters of the gas delivery assembly. While some of the physiological parameters that include, but not limited to temperature, blood pressure, blood glucose may be measured by the vitals sensor system and displayed on the user interface solely for assessment and monitoring purposes, at least one of the aforementioned physiological parameters measured by the VSS of the mask member is processed by the controller and determines at least one of the ventilation parameters/output of the gas delivery assembly.

The controller may be configured to calculate the respiratory rate, for instance, based at least partially on the intervals of tidal volume and/or capnographs readings and adjust/control the output of the gas delivery assembly accordingly. The volume of the gas per breathing cycle to be delivered by the gas delivery assembly to the patient may be calculated by the controller. For example, and without limitation, if a patient is breathing, the flow rate of inhaled/exhaled gas may be detected on exhalation/inhalation cycles of the patient and the duration of these cycles may be measured. The amount of gas to be delivered by the gas delivery assembly may be calculated at least partially based on a product of flow rate and time duration of the patient's breaths. If the patient is not breathing, the gas flow may be delivered by the gas delivery assembly until a pre-determined pressure is reached as measured by a pressure sensor. The system monitoring means may include at least one sensor configured to measure the oxygen content (FiO2) in the gas flow delivered to the patient that may be operatively connected to the mask body and/or the gas delivery assembly and wherein the FiO2 sensor(s) operatively connected to the controller. The necessary fraction of the oxygen in the gas to be delivered to the patient may be calculated by the controller and adjusted accordingly based, for instance, on the pulse oximeter readings and/or FiO2 readings. For example, and without limitation, the FiO2 sensor may be included in/connected to the inlet opening of the mask body.

In some embodiments, the gas delivery assembly may be configured to deliver intermittent input of the gas flow to the mask member, wherein the volume of the gas flow to the mask member is defined by the controller according to a logic circuit and/or an algorithm, based at least partially on the readings received from the tidal volume sensor. However, the care provider may adjust the parameters of the gas output from user interface when necessary.

The one-way inspiration valve operatively connected to the gas delivery assembly and the inlet portion of the mask member (e.g., to the inlet opening) and configured to allow the gas flow from the gas delivery assembly to the mask member and to prevent backflow. The inlet portion of the mask member may be configured to receive a gas delivery assembly and may include the one-way inspiration valve and/or membrane. In some embodiments, the inspiration valve may be embedded in the inlet opening. In some alternative embodiments, the inspiration valve may be located in the gas delivery assembly, for instance in the gas conduit. In some embodiments, the inspiration valve may be operatively connected to the controller and configured to open and close based on a command received by the controller; and wherein the controller configured to send the command to the inspiration valve.

The one-way expiration valve operatively connected to the outlet portion of the mask member (e.g., to the outlet opening) and configured to allow the gas flow from the mask member to the atmosphere and to prevent backflow; and wherein the valve operatively connected to the controller and configured to open and close based on a command received by the controller; and wherein the controller configured to send the command to the expiration valve. The outlet portion of the mask body includes the outlet opening configured to allow the gas flow from the mask member to the atmosphere, and wherein the outlet portion includes or connected to the one-way expiration valve/membrane. In some embodiments the expiration valve may be embedded in/connected to within the outlet opening.

In some embodiments, at least one of the inspiration and expiration valves may include other commercially available and custom designed valves and valves assemblies operable to open and close autonomously when a predetermined positive/negative pressure are reached within the mask member. For instance, one-way respiratory valves that permit the flow of gases in only one direction, such as check valves, PEEP valves, non-rebreather valves, but not limited to thereof. Other valves that are available for unidirectional respiratory circuits may be utilized; wherein the valves may be selected according to required resistance to flow or other parameters, and may include one or more of: a membrane, a filter, and condensate trap. Thus, in some embodiments at least one of the inspiration valve and expiration valve(s) may be not operatively connected to the controller, wherein the opening and closing of the valves functionality of the valves is relied upon the type of the valves structure/type and wherein the valve is operable to open/close when the pressure/vacuum reaches the desired value.

The system monitoring means operatively connected to the controller; wherein the means configured to at least transmit a signals to the controller, and the controller is configured to at least receive the signals from the system monitoring means; and wherein the system monitoring means further being operatively connected to at least one of the following: mask member, gas delivery assembly, at least one of the auxiliary modules; and wherein the controller configured to control and adjust the output ventilation parameters of the gas delivery assembly, control opening and closing function of the one-way expiration valve and may further control an output of at least one of the auxiliary modules, additionally according to signals received from at least one of the system monitoring means. The system monitoring means may include, but not limited to at least one of the devices including: a gas pressure sensor, a FiO2 sensor, a humidity sensor, a gas flow sensor, a gas temperature sensor, and any combinations thereof. In some embodiments, at least one gas pressure sensor may be operatively associated with and included in/connected to the mask member/body and configured to measure gas pressure within the mask body. The gas pressure sensor may be included in the mask body, for example but without limitation to the inlet and/or outlet portion of the mask body. In some embodiments, at least one gas flow sensor may be operatively associated with/or included in the mask member and configured to measure the flow rate of the gas entering and/or exiting the mask body. In some embodiments, the controller configured to open and close the expiration valve based at least on the signal received from at least one of the gas pressure sensors and/or flow sensors, when the signals reach the pre-set values. The gas flow sensor may be included in at least one of: mask body (e.g., the inlet portion of the mask body and/or the outlet portion of the mask body, but not limited thereto), a gas delivery assembly (e.g., in a gas conduit, but not limited thereto). In some embodiments, the gas pressure sensor and/or flow sensor may be an integral part(s) of the inspiration and/or expiration valves or valve assemblies. In some embodiments, at least one additional gas pressure sensor and/or gas flow sensor may be located elsewhere in the system, for instance in the gas delivery assembly. In some embodiments, the gas pressure sensor and/or flow sensor may be operatively connected to the expiration and/or inspiration valves; wherein the valves may include independent controlling units, and wherein the expiration and/or inspiration valves may be configured to open and close based on commands received from the units.

The FiO2 sensor is configured to measure oxygen concentration in the gas flow directed to the mask body, and may be located anywhere within the system, but preferably may be positioned in the inlet portion or connected to the inlet opening of the mask member but may be alternatively or additionally located in the gad delivery assembly, for instance in the gas conduit. The FiO2 sensor may be operatively connected to the controller.

The humidity sensor configured to measure the moisture content within the gas flow directed to the mask, may be located anywhere within the system, but preferably may be positioned in the inlet portion or connected to the inlet opening of the mask member and/or within gas conduit. The system comprising EVMA may further include at least one flow sensor operable to measure the rate of the gas flow to the mask that may be located in the gas delivery assembly, for instance, within the gas conduit; or be a part of a mask member, for instance, may be positioned within the inlet portion or connected to the inlet opening of the mask member. The humidity sensor may be operatively connected to the controller.

The gas temperature sensor may be located anywhere within the system, but preferably may be positioned in the inlet portion or connected to the inlet opening of the mask member and/or within gas conduit; and configured to measure the temperature of the gas delivered to the patient. The gas temperature sensor may be operatively connected to the controller.

In some embodiments, at least one of the system monitoring means may be configured to receive a signal from the controller, and the controller is configured to send the signals to the at least one system monitoring means.

In some embodiments, the system may include one or more auxiliary modules operatively connected to with the controller and the gas delivery assembly, wherein the one or more auxiliary modules comprising at least one auxiliary module selected from the group consisting of a humidifier, a dehumidifier, an oxygen source, an air/oxygen mixer, a nebulizer, an air heater, and any combinations thereof. At least one of the auxiliary modules may be configured to deliver additional therapeutic treatment to the patient through the gas flow generated by the gas delivery assembly, based on the commands received from the controller. In some embodiments, at least one of the auxiliary modules may be further operatively connected to at least one of the system monitoring means. The controller may be configured to control the output parameters of at least one auxiliary module, wherein the controller configured to send a control signals to auxiliary module and the auxiliary module configured to receive a control signal/command from the controller; and wherein the controller configured to control and adjust ventilation parameters of the gas delivery and the output parameters of at least one auxiliary module based at least partially on the data/signal associated with: at least two of the physiological parameters received from the vitals sensor system of the mask member; an input or the absence thereof from the user; and in some embodiments further based on signals received from the system control means. However, it is contemplated that in some embodiments at least one of the auxiliary modules is operatively connected to the gas delivery assembly, but not operatively associated with the controller, and configured to be operated by the user, wherein the output of the auxiliary module controlled by the user. The auxiliary modules may be fixedly or detachably coupled to the system, for instance to gas delivery assembly.

A system may further include one or more supporting modules, wherein the one or more supporting modules comprising at least one supporting module selected from the group consisting of an IV fluid warmer, suction pump, body fluids receptacle, and a storage unit, a medical refrigeration unit, and combinations thereof. The supporting modules may be fixedly or removably coupled to the system. In some embodiments, at least one of the supporting modules may be operatively connected to the controller, so that the controller configured to control setting parameters of the supporting modules where applicable. For instance, the controller may be able to control and adjust parameters of at least one of the following: temperature of IV fluid warmer, temperature of the medical refrigeration unit, operation of the suction pump.

In some embodiments, the system may be configured to receive/connect to an external oxygen source to the mask member. The mask body may include an oxygen port for receiving the oxygen source, wherein the port is configured to allow an oxygen flow to the mask body from an oxygen source. The oxygen source may be connected directly or through a conduit. The oxygen port may include a flow sensor and/or a regulator configured to display and adjust the flow rate of the oxygen to the mask body. In some embodiments, a flow sensor and/or a regulator may be operatively connected to the controller, and wherein the controller configured to control the rate of the oxygen delivery. In some embodiments, the oxygen port may further include an oxygen/air mixer, that may be operatively connected to the controller.

It some embodiments at least one of the auxiliary modules may be operatively connected to the mask member, and wherein the mask member may include an auxiliary port for receiving an auxiliary module that may be operatively connected to the controller or be configured to be controlled by the user. For example, and without limitation, the mask member may include an auxiliary port for receiving the nebulizer. In some embodiments the auxiliary port may include the oxygen port; while in other embodiments the auxiliary port and the oxygen port may be configured as separate ports.

While some of the parameters measured by the VSS of the mask member that may include but not limited to temperature, blood pressure, blood glucose, pulse, and combinations thereof may be measured by the vitals sensor system and displayed on the user interface solely for assessment and monitoring purposes, at least two of the physiological parameters measured by VSS may be processed by the controller and determine the output ventilation parameters of the gas delivery assembly and may further determine the output parameters of at least one of the following: one or more auxiliary modules, one or more supporting modules, a flow regulator of the oxygen port, a flow regulator of the auxiliary port.

The user interface (UI) operatively connected to the controller, the user interface comprising user alert means and user control means; wherein the user control means configured to receive an input from the user to the controller; and the user alert means configured to provide an output to the user from the controller. The UI configured to provide a visual and/or audible output to a user (e.g., care provider or patient when practical) and to receive a manual and/or voice input therefrom. The user control means configured to receive a manual and/or voice input from a user and may include but not limited to buttons, dials, levers, sliding knobs, touch/interactive screen, microphone, voice recognition means, such as software, remotely connected devices including, but not limited to mobile devices, computers, tablets and any combinations thereof, that are operable to enable a manual and/or voice and/or wirelessly transmitted command data input from the user. The user control means configured to receive the commands from the user associated with at least one of the following: the output ventilation parameters of the gas delivery assembly, output treatment parameters of auxiliary modules, parameters associated with controlling at least one of the supporting modules, gas flow parameters of an oxygen source coupled to the mask member, and any combinations thereof. In some embodiments, the user control means may be configured to receive the commands from the user associated with the VSS function, wherein the VSS is further configured to receive the input from the controller and the controller configured to send commands to VSS. The user alert means configured provide a visual and/or audible output to a user and may include but not limited to display, screen, color coded light, speaker, alarm, and any combinations thereof, that are operable to provide visual and/or audible output to the user. The user alert means configured to communicate data to the user associated with at least one of the following but not limited to: physiological parameters measured by the vitals sensor system of the mask member, the input received from the care provider, the output ventilation parameters of the gas delivery assembly, one or more parameters measured by the sensors/devices of the system monitoring means, output treatment parameters of auxiliary modules, gas flow parameters of an oxygen source coupled to the mask member, parameters associated with at least one of the supporting modules, and any combinations thereof. In some embodiments, the user interface may include a display and/or windows configured to display at least some of the aforementioned data. In some embodiments the user interface may include a display having a touch/interactive screen, operable to receive a manual input from the user, and that may be additionally operable to prove the visual output to the user. In some embodiments, the user alert means may be configured to provide audible voice command to the care provider. The user alert means may also be configured to provide visual cues to the care provider, that may include data representation in form of alphanumeric values, graphs, diagrams and/or continuous flashing light signals that may be color-coded. In some embodiments, the user alert means may include at least one data transfer module operable to transmit data to other devices, wherein the module includes but not limited to wired communication (ethernet, USB), Bluetooth device, Wi-Fi communication device, and proximity communication device, and any combinations thereof. The data transfer module may be particularly advantageous for transmitting the patient's data (e.g., data history of measured physiological parameters and/or received ventilation and/or auxiliary treatments) before or upon delivering the patient to a medical treatment facility and receiving patient medical records from a medical data storage facility. The user interface may be configured to include an input and output portions, wherein the input portion is configured to receive data from the controller and the output portion configured to send data to the controller; wherein the signals/data may be transferred directly or wirelessly.

In some embodiments, the system user alert means of the user interface may provide visual and/or audible alerts based at least on the capnograph readings. For example, and without limitation, when capnograph determines that partial pressure of CO₂ deviates from a known capnography waveform of a healthy individual, which may be indicative for example and without limitation of respiratory acidosis/alkalosis, bronchospasm, the system may alert the care provider to attach an external oxygen source or nebulizer to increase oxygen concentration in the gas flow by engaging the auxiliary oxygen source coupled to the gas delivery assembly or attaching the nebulizer to deliver aerosolized medication. The readings from the capnograph can also be used to confirm proper endotracheal tube placement. In some embodiments, visual alerts may be alternatively or additionally shown on the mask member; for example, and without limitation the mask member may include an alert portion operatively connected to at least one sensor of the VSS and/or at least one of the system monitoring means, wherein a color-coded alerts may be displayed. In some embodiments, the alert portion may be connected to the controller.

In some embodiments, the system user alert means may provide visual and/or audible alerts based at least on the pulse oximeter readings. For example, when SpO2 reading fall below the pre-determined value, it alerts provider to attach an external oxygen source to the mask member port or to increase oxygen concentration in the gas flow by engaging the auxiliary oxygen source coupled to the gas delivery assembly. In some embodiments, the SPO2 value is kept in the range of 80-100%, but preferably 90-100%.

The system is configured to be connected to a source of power, for instance, to a source of electrical power. In some embodiments, the system may further include interchangeable power supply having an input portion and a discharge portion; and wherein the controller operatively connected to the discharge portion of the power supply. However, any parts and components of the system may be configured to be connected to a source of power and/or the power supply individually or through one or more of the components that in its/their term may be configured to be connected to a power source/power supply. In some embodiments, the system may include a battery, that may be rechargeable; and wherein the battery may be fixedly or detachably mounted to the system; for example, but without limitation, to the gas delivery assembly. In some embodiments, the system may additionally include a solar panel that may be operatively connected to a battery.

The system comprising EVMA may be configured to operate in the CPR sub-mode, wherein the CPR sub-mode may be switched into manually by provider or initiated automatically be the controller when no pulse and no breathing detected by the sensor system. In such a scenario, EVMA alerts the provider (visually and/or auditory) to initiate CPR and may give prompts for chest compression intervals and will automatically deliver rescue breaths when necessary. The CPR mode may be activated by the controller at least based on the absence of pulse detected by the heart rate sensor of the VSS. For example, and without limitation, the system may assist the care provider in performing CPR by providing counts for chest compressions and delivering rescue breaths and repeating the cycle until pulse is detected or system is manually switched out of CPR sub-mode by care provider.

The system comprising EVMA may be configured to operate in the non-rebreather sub-mode, when a patient is not required assisted ventilation, but still requires supplemental oxygen. In this scenario, the system may enter the non-rebreather mode either by manually input by the provider or automatically according to at least one parameter received form at least one sensor of the vitals sensor system; for instance, but not limited to tidal volume, breathing rate, oxygen saturation SpO2, PEEP or any combination thereof, and according to pre-determined algorithm. In this scenario, oxygen from an external source is provided from the auxiliary module to the gas delivery assembly or directly to the mask member, while the expiration valve(s) of the mask member remain open. This will allow for administration of oxygen to the patient that does not require ventilation assistance while the at least some of the physiological parameters still may be measured by the vitals sensor system and displayed at the user interface.

Each of the aforementioned and forgoing implementations, features, elements and steps of the methods may be employed individually or in conjunction and may include one or more of the aforementioned and following features, elements and steps in any suitable combination.

For instance, once the mask member is properly secured on the patient, and at least one sensor of the sensor system receive a signal indicating that patient is breathing on her/his own and that pulse is present; the breathing pattern data, such as tidal volume and breathing rate, and heart rate/pulse, data are sent to the controller that sets and continuously adjust ventilation parameters of the gas delivery assembly (such as volume and frequency of gas output) based on the most recent data received from the sensor system according to predetermined algorithm. The breathing pattern may be assessed by the tidal volume sensor and/or capnograph that may be connected to the expiration valve. Pulse may be assessed, for instance by the pulse oximetry sensor and/or blood pressure sensor or combination thereof.

If no breathing pattern and pulse are detected by the sensor system and no data received by the controller, the system will initiate CPR mode. The CPR mode may be switched on manually by the provider or initiated automatically be the controller. In such a scenario, EVMA of the system may alert the provider (visually and/or auditory) to initiate CPR and may give prompts for chest compression intervals and will automatically deliver rescue breaths when necessary.

If pulse is present but no breathing is detected, a manual override may be initiated; that is a care provider sets ventilation parameters, and gas delivery assembly operates based on the manually input ventilation parameters. When/if the breathing is resumed as detected by the sensor system, the system alerts the provider that breathing is resumed and provides a prompt that allows the provider to choose between continuing with manual settings or switching to an automatic mode. If/when an automatic mode is selected, the controller will set and continuously adjust ventilation parameters based on the sensor system reading according to the pre-determined algorithm.

Furthermore, in any/all aforementioned scenarios, if any of the parameters measured by the sensor system fall out of normal (pre-set range) the alert will be given to the provider, that may be visual, audible, or both, and that may be further displayed by user interface.

Additional advantages of the invention will be set forth in part in the description that follows, and in part will be obvious from the description, or may be learned by practice of the invention. It is to be understood that both the forgoing general description and the following detailed description are exemplary and exemplary only and are not restrictive of the invention as described and claimed.

The VSS may include at least one sensor configured to measure at least one of the following parameters: temperature, blood pressure, blood glucose level, heart rate, oxygen blood saturation (SpO2); partial pressure of carbon dioxide in the exhaled breath, positive end-expiratory pressure (PEEP), tidal volume, cardiac function, and any combinations thereof. The VSS may further include at least one vapor sensor, configured to measure gas composition. For instance, the vapor sensor may be configured to measure ethanol, ketones, or other substances.

For instance, the photoplethysmography (PGG) sensors may be also used for measuring oxygen saturation, blood pressure, heart rate, respiratory rate and cardiac output assessing autonomic function and detecting peripheral vascular disease; wherein forehead-type PGG devices may be used. However, with a proper calibration alternative facial/head areas may be utilized. Therefore, the sensors for measuring oxygen saturation, blood pressure, heart rate, respiratory rate and cardiac output assessing autonomic function and detecting peripheral vascular disease, may be advantageously positioned in the glabellar portion of the mask member. In some embodiments the glabellar portion may be further extended and/or expanded to cover the larger portion of the forehead area to accommodate for the plurality of sensors/devices as needed.

Temperature may be measured by a thermal energy transfer sensor that detects heat moving from or into the body and/or forehead thermometer. These are perceived as the least disruptive and therefore the most comfortable way to measure body temperature. Temperature sensors may include: single thermopile sensors; thermopile array sensors, infrared thermometers; thermal cameras; NTC (negative thermal coefficient) thermistors, the temperature system sensors (TSYS), thermocouples, RTDs (resistance temperature detectors), thermistors, semiconductor based integrated circuits (IC), negative temperature coefficient (NTC) sensor for body temperature.

The blood pressure may be measured, for example by devices/sensors including photoplethysmography (PPG) sensors, transdermal optical imaging (TOI), dual photoplethysmography (PPG) sensors, digital optical sensors, optical emitters, optical detectors, photodetectors, RBG cameras that may be used in conjunction with non-magnifying signal processing approaches, infrared cameras that may be used in conjunction with motion magnification, near-infrared and any combinations thereof.

Blood glucose level can be measured using electrochemical, optical, and electromagnetic/microwave non-invasive blood glucose monitoring techniques or microwave dielectric spectroscopy techniques.

Heart rate/pulse can be measured by electrical or optical sensors.

Oxygen blood saturation (SpO2) may be measured by a pulse oximeter based on a transmission or reflectance probes.

A partial pressure of carbon dioxide CO₂ in the exhaled breath may be measured by a capnograph.

A tidal volume may be measured by a flow sensor including differential pressure flow meter, rotameter, orifice flow meter, venturi flowmeter or positive displacement flow meter.

The stroke volume may be measured by FloTrac sensor that uses arterial pressure waveform analysis and calculations, or other sensor relying on the arterial waveform analysis and/or doppler ultrasound flow sensor. In some embodiments, the VSS of the mask member may include at least one sensor/device configured to measure at least one cardiac function, such as stroke volume and/or cardiac output and/or arterial fibrillation detection, wherein the cardiac output is the volumetric flow rate of the heart's pumping output, and which may be the product of the heart rate, and the stroke volume, which is the volume of blood pumped from the left ventricle per beat.

The mask member may further include at least one removable mask liner configured to provide a barrier between the at least partial facial area of a patient and the inner surface of the mask member, and wherein the liner material is not interfering with ability of sensors of VSS to provide accurate measurements. The liners may be disposable or reusable and made at least partially from a material that may be sterilized.

The mask member may further include at least one removable mask liner configured to provide a barrier between the at least partial facial area of a patient and the inner surface of the mask member, and wherein the liner material is not interfering with ability of sensors of VSS to provide accurate measurements. The liners may be disposable or reusable and made at least partially from a material that may be sterilized.

### BRIEF DESCRIPTION OF THE DRAWINGS

The application contains 20 drawing sheets containing 16 figures depicting some of the exemplary embodiments of the invention.

The figures represent some exemplary configurations of the present invention and some exemplary methods of using thereof. All features and functionalities that are depicted are not to be considered to be entirely bound to the particular configuration and can be included in any combination in any other embodiment of the present invention. Furthermore, all shapes and dimensions of embodiments as whole and all dimensions, shapes and patterns of its depicted features, are not limiting and serve for an exemplary purpose only. The embodiments and all their features, parts, dimensions and patterns are not depicted to scale.

FIG. 1 is a block diagram of an emergency ventilating and monitoring system comprising an emergency ventilating and monitoring apparatus (EVMA) in accordance with an exemplary embodiment of the invention.

FIG. 2A is a front view of an emergency ventilating and monitoring system comprising an EVMA in accordance with an exemplary embodiment of the invention, and wherein a gas conduit is shown in extended position.

FIG. 2B is a front view of an emergency ventilating and monitoring system comprising an EVMA in accordance with another exemplary embodiment of the invention, and wherein a gas conduit is shown in extended position.

FIG. 3 is a side view of the systems illustrated in FIGS. 2A-B, wherein the gas conduit is shown in extended position.

FIG. 4 is a side view of the systems illustrated in FIG. 2A-B and FIG. 3, wherein the gas conduit is shown to be retracted to a conduit management assembly.

FIG. 5A is a side view of a mask member according to an exemplary embodiment of the invention, wherein the mask member is depicted to be worn by a subject.

FIG. 5B is a side view of a mask member according to another exemplary embodiment of the invention, wherein the mask member is depicted to be worn by a subject.

FIG. 5C is a side view of a mask member according to yet another exemplary embodiment of the invention, wherein the mask member is depicted to be worn by a subject.

FIG. 6 is a perspective view of the mask member illustrated in FIG. 5A.

FIG. 7 is a front view of the mask member illustrated in FIG. 5A.

FIG. 8 is a cross-sectional view on the line A-A of the mask member illustrated in FIG. 6.

FIG. 9 is a back view of the mask member illustrated in FIG. 7.

FIG. 10 is a perspective view of a pump member according to an exemplary embodiment of the invention.

FIG. 11 is a front view of the pump member illustrated on FIG. 10.

FIG. 12A is a cross-sectional view on the line B-B of the pump member illustrated in FIG. 10 and FIG. 11.

FIG. 12B is a cross-sectional view on the line C-C of the pump member illustrated in FIG. 10.

FIG. 13 is a front view of a gas conduit according to an exemplary embodiment of the invention, and a partial cross-sectional view on the line D-D.

FIG. 14 is a perspective view of a conduit management assembly according to an exemplary embodiment of the invention, wherein the conduit management assembly is shown bent about a knee.

FIG. 15A is another perspective view of the conduit management assembly illustrated in FIG. 14, wherein the conduit management assembly is shown in a straightened position.

FIG. 15B is a perspective view of a conduit management assembly according to another exemplary embodiment of the invention, wherein the conduit management assembly is shown in a straightened position.

FIG. 16 is an exploded view of the conduit management assembly illustrated in FIG. 14 and FIG. 15A.

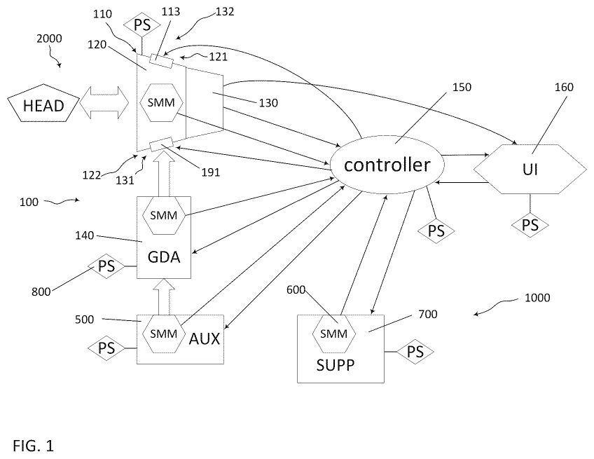

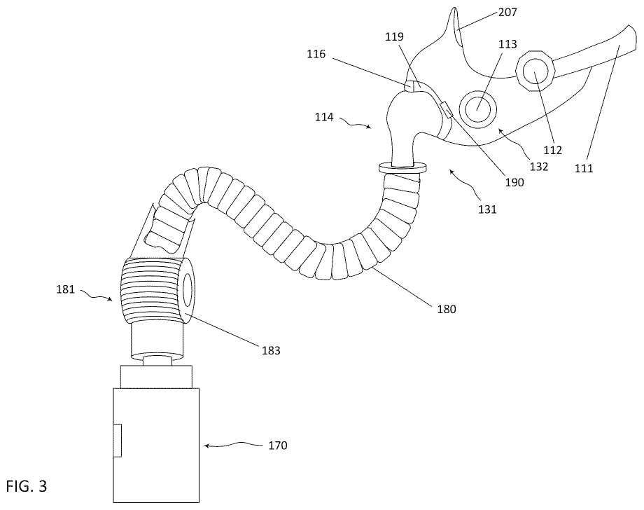

The following indicia are used in FIGS. 1-16 and throughout the entirety of the disclosure to facilitate understanding of embodiments of the invention: emergency ventilating and monitoring system 1000; patient/subject head 2000; emergency ventilating and monitoring apparatus (EVMA) 100; auxiliary modules (AUX) 500; system monitoring means (SMM) 600, supporting modules (SUPP) 700; power source 800; mask member 110; head band 111; head band fastener 112; expiration valve 113; gas intake hatch 114; conduit hub 115; hinge 116; chute 117; window 118; seal bands 119; mask body 120; outlet opening 121; inlet opening 122; inner surface 123; outer surface 124; glabellar portion 125; nasal portion 126; frontal portion 127; mandibular portion 128; temporal portion 129; vitals sensor system (VSS) 130; inlet portion of the mask member 131; outlet portion of the mask member 132; gas delivery assembly (GDA) 140; controller 150; user interface (UI) 160; pump member 170; jacket 171; upper section 172; lower section 173; charging port for battery 174; drum 175; outer surface of the drum 176; drum cavity 177; drum roof 178; gas duct 179; gas conduit 180; conduit management assembly 181; knee 182; tube 183; housing 184; bores 185; spine 186; spine ribs 187; upper clip 188; lower clip 189; hatch release lock 190; inspiration valve 191; pressure sensor 192; tidal volume sensor 193; capnograph 194; PEEP monitor/sensor 195; temporal pulse sensor 196; mandibular pulse sensor 197; fluid detector 198; nasal sensor pad 199; thermometer 200; pulse oximeter 201; blood pressure monitor 202; blood glucose sensor 203; glabellar area of sensor pad 204; nasal area 205 of sensor pad 199; lining 206; lining bar 207; piston hub 208; piston assembly 209; piston actuator 210; piston 211; leg 212; plunger 213; inlet port 214; lower pump valve 215; filter 216; gas channel(s) 217; outlet port 218; upper pump valve 219; jacket inner wall 220; pump terminal 221; upper guide 222; lower guide 223; upper spout 224; lower spout 225; window 226; inner hoops 227; extrusions 228; outer hoops 229; divots 230; oxygen and/or auxiliary port 231, occipital portion of the mask member 232; VSS pad 233; clip 234; band 235.

### DETAILED DESCRIPTION

Those of ordinary skill in the art realize that the following descriptions of the embodiments of the present invention are illustrative and are not intended to be limiting in any way. Other embodiments of the present invention will readily suggest themselves to such skilled person having the benefit of this disclosure. Like numbers refer to like elements and intended to be consistent throughout. Although the following detailed description contains many specifics for the purposes of illustration and facilitation of understanding of the invention, anyone of ordinary skill in the art will appreciate that many variations and alterations to the following details are within the scope of the invention. Accordingly, the following embodiments of the invention are set forth without any loss of generality to, and without imposing limitations upon the scope of current disclosure.

In this detailed description of the present invention, a person skilled in the art should note that directional terms, such as “above”, “below”, “upper”, “lower”, “proximal”, “distal”, “front”, “back”, and other like terms, are used for convenience of the reader in reference to the drawings. Also, a person skilled in the art should notice that this description may contain other terminology to convey position, orientation, and direction without departing from the principles of the present invention. Furthermore, in this detailed description, a person skilled in the art should note that quantitative qualifying terms such as “generally”, “substantially”, “mostly”, and other like terms are used, in general, to mean that the referred to object, characteristic, or quality constitutes a majority of the subject of the reference. The meaning of any of these terms is dependent upon the context in which it is used, and meaning may be expressly modified.

Reference will now be made in detail to the present exemplary embodiments, examples of which are illustrated in the accompanying drawings FIGS. 1-16. It should be understood that the invention is not limited to these disclosed embodiments. Likewise, while “the invention” or “present invention” may have been referred to at times in this disclosure, those terms are not intended to limit the scope of this disclosure or suggest in any way that this is a single version or embodiment. While the invention relates generally to the Emergency Ventilating and Monitoring System, the systems that fall within the scope of this disclosure may include a variety of optional features, which do not need to be present in every version or embodiment. Various modifications and changes can be made without departing from the spirit of the invention. In fact, modifications, customizations, and other embodiments of the invention will also come to mind of those skilled in the art having the benefit of current disclosure, and which are intended to be and are covered by current disclosure. The System and EVMA of this invention, however, may be embodied in many different forms and should not be construed as limited to the embodiments set forth herein. Rather, these embodiments are provided so this disclosure will be thorough and complete and will fully convey the scope of the invention to those skilled in the art.

Mentioning one or more representative features of a given embodiment is likewise exemplary. Such an embodiment can typically exist with or without features mentioned; likewise, those features can be applied to other embodiments of the presently disclosed subject matter.

Referring to FIG. 1, the diagram shows an emergency ventilating and monitoring system 1000 comprising an emergency ventilating and monitoring apparatus (EVMA) 100 in accordance with an exemplary embodiment of the invention, wherein exemplary configuration of functional interconnection between modules and components. As shown, the system 1000 may further include at least one of the auxiliary modules 500 and at least one of the supporting modules 700. The EVMA 100 comprising a mask member 110 having a mask body 120, a vitals sensor system (VSS) 130 operatively connected to the mask member 110, wherein the mask body 120 having an outlet portion 132 including an expiration valve 113 and an inlet portion 131 including an inspiration valve 191, a gas delivery assembly 140 operatively connected to the mask member 110, the controller 150, the user interface 160, and the system monitoring means 600. The vitals sensor system (VSS) 130 operatively connected to the mask member 110 and comprising one or more sensors configured to measure signals associated with one or more physiological parameters of a patient; and wherein the one or more physiological parameters comprising at least one physiological parameter selected from the group consisting of: temperature, blood oxygen saturation (SpO2), heart rate, blood pressure, blood glucose level, partial pressure of carbon dioxide in the exhaled breath, positive end-expiratory pressure (PEEP), tidal volume, cardiac function, and combinations thereof. In some embodiments, the mask member 110 may further include fastening means (not shown) configured to secure the mask body 120 on the head of the subject 2000. In some embodiments, the mask member 110 may include a chin support (not shown), that may be included in the mask body 120.

The vitals sensor system 130 operatively connected to the controller 150, and wherein the controller 150 configured to receive and process signals from VSS 130. The user interface 160 is configured to make at least the data measured by VSS 130 available to a care provider ether directly or through the processor 150. The controller 150 operatively connected to the user interface 160, the expiration valve 113, the gas delivery assembly 140, system monitoring means 600, and may be operatively connected to the inspiration valve 191, the auxiliary modules 500, and supporting modules 700.

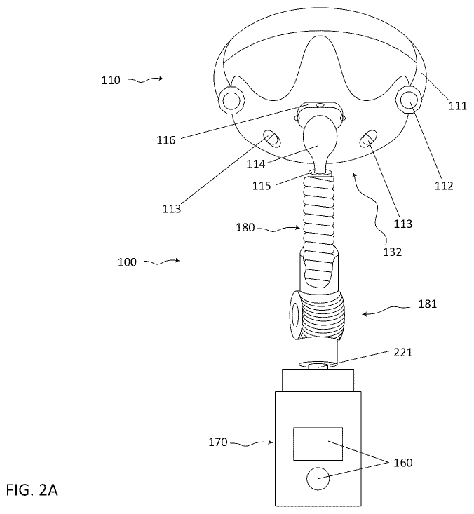

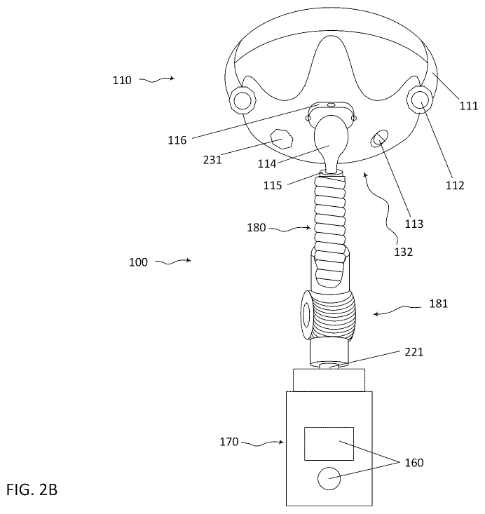

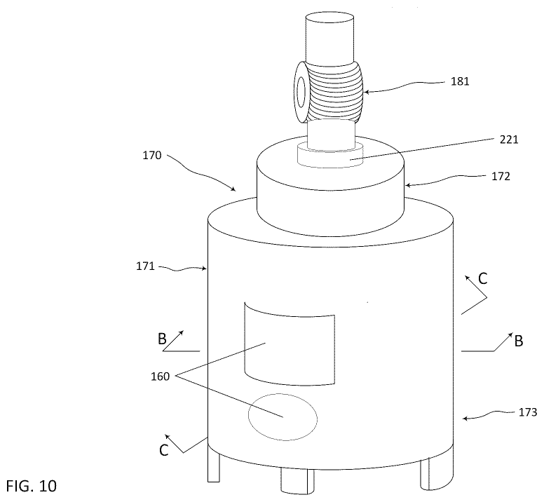

The mask member 110 includes the mask body 120 configured for engagement with at least partial facial/head area of a subject which includes an outlet portion 132 having the outlet opening 121 and an inlet portion 131 having inlet opening 122 extending between the inner and the outer surfaces of the mask body 120. The inlet portion 131 is configured to receive a gas delivery assembly 140 and includes the inspiration valve 191 configured to allow the gas flow from the gas delivery assembly 140 to the mask body 120 and to prevent backflow. In some embodiments, the inspiration valve 191 may alternatively be a part of/connected to the gas delivery assembly 140; for example, and without limitation, the inspiration valve 191 may be positioned in the gas conduit or the pump (exemplary embodiments thereof are shown on FIGS. 2, 10,11,12,13). The outlet portion 132 includes the outlet opening 121 extending between the inner and the outer surfaces of the mask body 120. The outlet portion 132 includes a one-way expiration valve 113 and/or membrane (not shown) configured to allow a gas flow from the mask body 120 to the atmosphere and to prevent backflow, and that is operatively connected to the controller 150 and configured to open and close based on the command received from the controller 150. The expiration valve 113 may be configured to remain closed when gas flow is being delivered to the mask body 120 from the gas delivery assembly 140, based on a command received from the controller 150.

The system configured to be connected to a source of power and may further include interchangeable power supply (not shown) having an input portion and a discharge portion. In some embodiments, the controller 150 may be operatively connected to the discharge portion of the power supply. However, any component of the system may be configured to be operatively connected to the source of power or the power supply, wherein at least some of the components may be operatively connected to the source of power/power supply independently or through other components.

The gas delivery assembly 140 operatively connected to the inlet portion 132 of the mask body 120 and configured to generate and direct gas flow to the inlet portion 132 of the mask body 120. The gas delivery assembly 140 may include at least one of the devices such as a pump, a blower, a compressible bag (for instance, self-inflating bag), and combinations thereof; or other devices known in the art that are operable to deliver directed intermittent and/or continuous gaseous output.

The controller 150 is positioned operatively connected to the gas delivery assembly 140, the vitals sensor system 130 of the mask member 110, and configured to receive the signals associated with a plurality of physiological parameters of the patient from the vitals sensor system 130 of the mask member 110, process the signals, and control the the gas flow output of the gas delivery assembly 140 based at least partially on the signals associated with at least one of the physiological parameters received from the vitals sensor system 130 of the mask member 110. The user interface 160 operatively connected to with the controller 150, wherein the user interface 160 comprising user alert means and user control means; wherein the user control means configured to receive an input from the user to the controller 150, and the user alert means configured to provide an output to the user from the controller 150; and wherein the controller 150 further configured to control the output of the gas delivery assembly 140 based at least partially on the input from the user. The controller 150 may further include a memory/data storage module operable to store the measured data received from the vitals sensor system 130 and/or reference data for at least one parameter measured by the vitals sensor system 130. For example, and without limitation, the controller 150 may include one or more of the following: a logic circuit, a computer, a processor, memory, and any combinations thereof.

The one-way inspiration valve 191 is operatively connected to the gas delivery assembly 140 and the inlet portion 131 of the mask body 120, and in some embodiments may be included in the inlet portion 131 of the mask body 120; the inspiration valve 191 configured to allow the gas flow from the gas delivery assembly 140 to the mask body 120 and to prevent backflow. In some embodiments, the inspiration valve 191 may be operatively connected to the controller 150 and configured to open and close based at least partially on a command received from the controller 150.

At least one one-way expiration valve 113 operatively connected to the outlet portion 132 of the mask body 120 and configured to allow the gas flow from the mask body 120 to the atmosphere; and wherein the expiration valve 113 operatively connected to the controller 150 and configured to open and close based at least partially on a command received from the controller 150.

At least one system monitoring means 600 is operatively connected to the controller 150, and to at least one of the following: the mask member 110, the gas delivery assembly 140; and wherein the means 600 configured to at least send a signal to the controller 150; and wherein the controller 150 further configured control the ventilation parameters of the gas delivery assembly 140 and the operation of the one-way expiration valve 113 based at least partially on a signal received from at least one of the system monitoring means 600. In some embodiments, the system monitoring comprising at least one sensor selected from a group consisting of: a pressure sensor, a temperature sensor, a FiO2 sensor, a humidity sensor, a flow sensor, a vapor sensor, and any combinations thereof. The controller 150 may be further configured to receive the data from and/or control the system monitoring means 600 according to pre-determined algorithms and may further be operable to store the measured data received from the means 600 and/or reference data for at least one parameter measured by the system monitoring means 600 and/or parameters measured by VSS 130. The system monitoring means 600 may include at least one system monitoring means selected from the group consisting of: a pressure sensor operatively connected to the mask member 110 and configured to measure gas pressure within the mask body 120, a flow sensor operatively connected to the mask member 110 and configured at least to measure a gas flow exiting airways of the patient, and combinations thereof. For example, and without limitation, the FiO2 sensor may be connected to the inlet portion 132 of the mask member 110 or be positioned within the gas delivery assembly 140; for instance, within gas conduit, but not limited thereto. In some embodiments, the system monitoring means 600 may include at least one flow sensor operatively connected to the mask body 120 and/or gas delivery assembly 140 and configured to measure at least one of the following: a flow rate of the gas entering the mask body 120, a flow rate of the gas exiting the mask body 120.

The system 1000 may further include one or more auxiliary modules 500 operatively connected to with the controller 150 and the gas delivery assembly 140; and wherein the one or more auxiliary modules 500 comprising at least one auxiliary module selected from the group consisting of a humidifier, an oxygen source, an air/oxygen mixer, a nebulizer, an air heater, a filter (for instance, HEPA filter but not limited thereto), and combinations thereof. For example, and without limitation, the nebulizer can be used to administer additional therapies to the patient via the gas flow base on the reading from the vitals sensor system 130 and/or according to provider's expertise and instructions. For example, and without limitation, the system monitoring means 600 may include a sensor configured to measure moisture content within the gas flow, that may be in communication with the controller 150, and wherein controller 150 may be configured to adjust the moisture content within the gas flow delivered to the mask body 120 according to the pre-determined algorithm and/or user input, and wherein the additional moisture/humidity may be supplied by the auxiliary humidifier.

The system 1000 may include one or more supporting modules 700, wherein the one or more supporting modules 700 comprising at least one supporting module 700 selected from the group consisting of: an IV fluids warmer, suction pump, body fluids receptacle, and a storage unit, a refrigeration/freezer unit, and combinations thereof. In some embodiments, at least one of the supporting modules 700 may be operatively connected to the controller 150.

The controller 150 may be configured to continuously and/or intermittently receive, collect and record signals measured by the vitals sensor system 130 of the mask member 110 and process them according to predetermined algorithm. Then, based at least on the most recent signal output from the vitals sensors system 130, the controller 150 may be operable to generate/adjust ventilation parameters of the gas delivery assembly 140 (for instance, of the pump member), so the gas delivery assembly 140 generates and delivers the appropriate gas flow to the mask member at any given breathing cycle. For example, and without limitation, the controller 150 may include a logic circuit that may be configured to calculate lungs volume of the patient and thus, the necessary volume of the gaseous output to be delivered by the gas delivery assembly 140 based, for example and without limitation, on the signal received from the tidal volume sensor of VSS 130; and may be configured to calculate the respiratory rate of the patient based on the intervals that tidal volume sensor and/or capnographs receives readings, and thus, establish/adjust the frequency with which the gas delivery assembly 140 needs to generate the gas flow output. Moreover, the input from the SpO2 sensor may be processed by the controller 150, and wherein the data may be indicative that the patient blood oxygen saturation is lower than pre-determined value, the controller 150 may send the command to the auxiliary oxygen source module to increase the concentration of the oxygen (FiO2) in the gas flow directed to the mask member 110. The controller 150 may repeat this command until the SpO2 reading of the patient stabilizes within a normal predetermined range.

The user interface 160 operatively connected to the controller 150, wherein the user interface 160 comprising user alert means and user control means; wherein the user control means configured to receive an input from the user to the controller, and the user alert means configured to provide an output to the user from the controller; and wherein the controller 150 further configured to control the ventilation parameters of the gas delivery assembly 140 based at least partially on the input from the user. In some embodiments, the user interface 160 may be configured to display parameters/data received from the at least some of the system control means 600 including a pressure sensor, a temperature sensor, a FiO2 sensor, a flow sensor, a humidity sensor, a vapor sensor, and combinations thereof.

Referring to FIGS. 2-16, the diagrams are shown illustrating various views and cross sections of exemplary embodiments of an emergency ventilating and monitoring apparatus (EVMA) 100 and its exemplary components in accordance with exemplary embodiments of the system 1000.

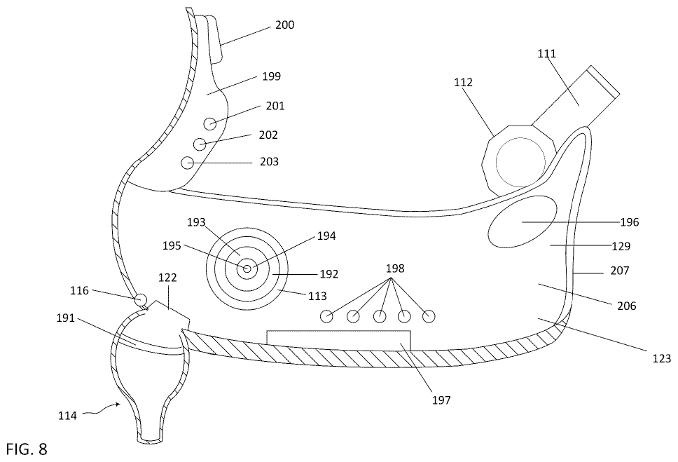

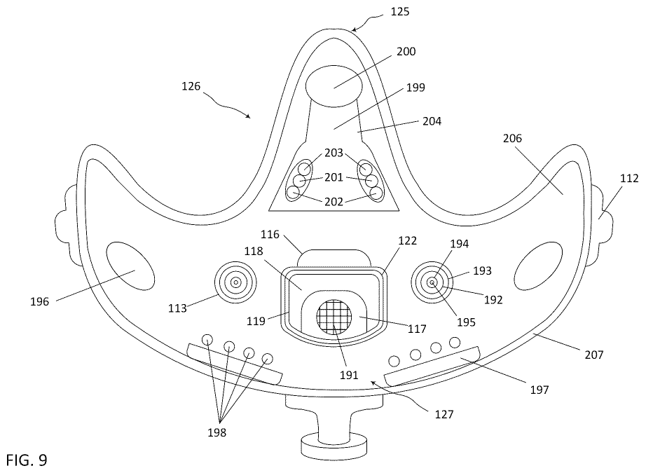

As shown on FIGS. 2-4, a system comprising EVMA includes a mask member 110 having a mask body 120, and a gas delivery assembly 140, a controller schematically depicted as 150, and a user interface schematically depicted as 160. The gas delivery assembly 140 may include at least one gas delivery unit, that in some embodiments may include a pump member 170, and may further include a gas conduit 180. The gas conduit 180 positioned between the pump member 170 and the mask body 120 and configured to direct gas flow from the pump member 170 to the mask body 120. In some embodiments, the pump member 170 is configured to deliver an intermittent output of gas into the mask body 120. The vitals sensor system (VSS) is not shown herein and is schematically depicted on FIGS. 1 as 130, and exemplary embodiments thereof are best shown on FIGS. 8-9. The controller 150 is positioned operatively connected to the pump member 170 and the vitals sensor system of the mask member 110, wherein the controller 150 is configured to control ventilation parameters of the pump member 170 at least partially based on at least one physiological parameter measured by the VSS of the mask member 110. The user interface 160 is positioned operatively connected to with the controller 150. The precise positions of the controller 150 and a user interface 160 are not particularly limited. In some embodiments, the controller 150 and/or the user interface 160 may be connected to or imbedded in the pump member 170. However, configuration wherein the controller and/or user interface may be in whole or partially connected to/included in the mask member 110 and/or elsewhere gas delivery assembly are contemplated and included in the scope of the disclosure. The mask member 110 may include fastening means. The fastening means may include a head band 111, that in some embodiments, may be pivotally connected to the mask body 120 and configured to secure the mask body 120 to the head of the patient 2000; for instance, with aid of the head band fastener 112, wherein the fastener 112 may be configured to adjust/tighten the head band 111 around head of the patient. The design of the fastening means and the head band fastener 112 are not particularly limiting, and may include elastic straps, and other fasteners known in the art. In some embodiments, the head band 111 may configured to fasten automatically around the patient's head; for instance, once the controller determines that the mask member has been applied to the of the patient; for instance via at least one of the following: contact sensor, pressure sensor, proximity sensor; and wherein the mask member includes at least one the aforementioned sensors, that may be connected to the mask body (e.g., to the inner surface of the mask body) and/or to the fastening means, such as the head band, but not limited thereto. The operation of the fastening means may be additionally or alternatively controllable through the user interface input. The depicted mask member and gas delivery assembly designs are advantageous, but exemplary and not limiting.

As shown, the mask member 110 includes the mask body 120, wherein the mask body has an inlet portion 131 including an inlet opening 122, and an outlet portion 132 including an outlet opening 121. The mask body 120 includes a one-way inspiration valve 191 operatively connected to the gas delivery assembly 140 and the inlet portion 131 of the mask body 120 and configured to allow the gas flow from the gas delivery assembly 140 to the mask body 120 and prevent backflow. The inspiration valve 191 may be included in the inlet opening 122 or be positioned elsewhere within the inlet portion 131. The inlet portion 131 of the mask body 120 may be configured to receive the gas delivery assembly 140 and may include the one-way inspiration valve/membrane 191 (shown on FIG. 9). In some embodiments, the inspiration valve may be operatively connected to the controller 150.

The one-way expiration valves 113 operatively connected to the outlet portion 132 of the mask body 120 and configured to allow the gas flow from the mask member to the atmosphere, wherein the expiration valve operatively connected to the controller 150 and configured to open and close based on the command received from the controller 150. Though, two expiration valves 113 are depicted herein, in some embodiments, the mask member may include a single outlet portion 132 having a single expiration valve 113. The expiration valve(s) 113 may be configured to remain closed when gas flow is being delivered from the gas delivery assembly 140 to the mask body 120, based on the command from the controller 150.

The gas delivery assembly 140 may include a pump member 170 operatively connected to with the controller 150, and a gas conduit 180 extending between the pump member 170 and the inlet portion 131 of the mask member 110 and configured to direct the gas flow from the pump member 170 to the inlet portion 131 of the mask body 120. The gas delivery assembly 140 may further include a conduit management assembly 181. The conduit management assembly 181 may be fixedly connected to the pump member 170 or mask member 110; or may be configured to detachably couple to the mask member 110 and/or the pump member 170 and configured to at least partially receive/enclose at least a portion of the gas conduit 180.

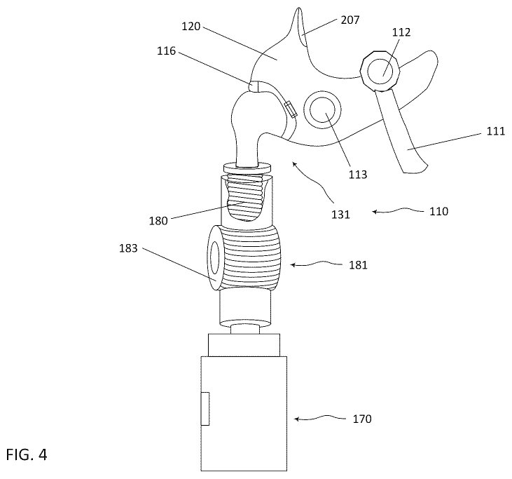

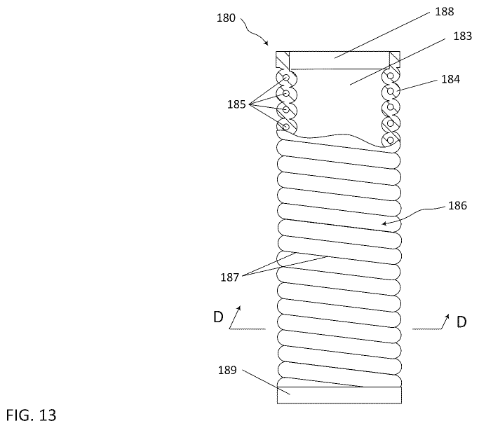

As shown on FIG. 4, the conduit management assembly 181 may be configured to receive an entirety of the contracted gas conduit 180. However, it is contemplated and included in the scope of the disclosure that the conduit management assembly 181 may be configured to receive a portion of the gas conduit 180, or selectively receive a portion of the gas conduit 180, so that the length of the gas conduit 180 may be adjustable according to the user needs and height of the patient. The conduit management assembly 181 may further include a knee 182, that enables the bending of the conduit assembly 181, without substantially pinching the gas conduit 180, that passes through. The conduit management assembly 181 reduces the possibility of gas conduit pinching, and may further allow to adjust the length of the gas conduit as necessary, thus reducing chances of entanglement of the gas conduit 180 with other objects; and making the system more compact for storage and transport. Exemplary configurations of the gas conduit 180 and the conduit management assembly 181 are shown in more detail in FIGS. 13-16.

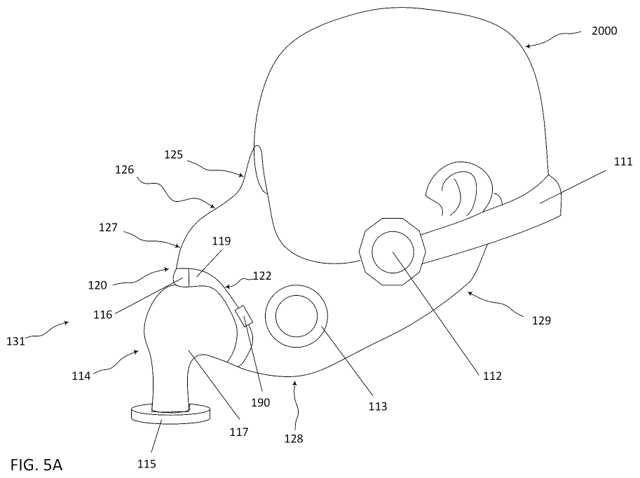

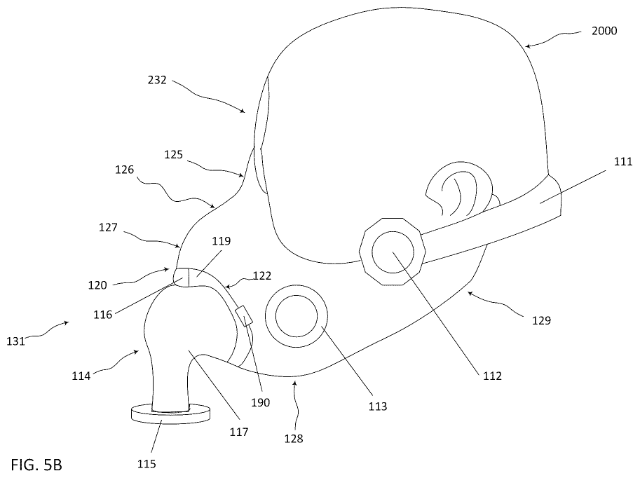

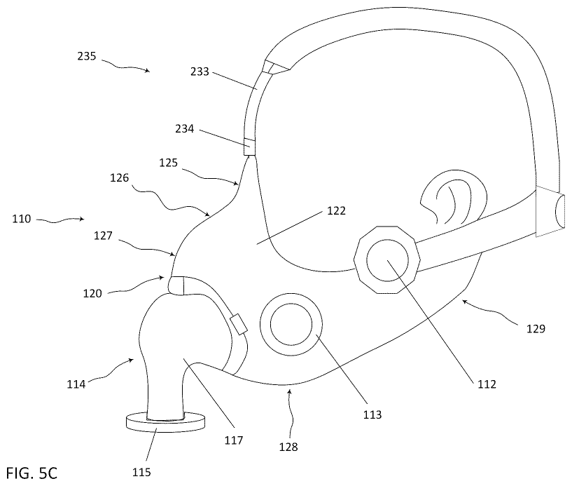

Referring now to FIG. 5-9, an exemplary implementation of the mask member 110 according to an exemplary embodiment of the invention is now described in greater detail. The FIG. 5 depicts the mask member 110 being worn by a patient 2000. As shown, the mask member 110 comprises a mask body 120 and a head band 111. The mask body 120 includes an outer surface 124 and an inner surface (shown on FIGS. 8-9 as 123) configured for engagement with face and/head of the patient.

The mask body 120 may include a glabellar portion 125, a nasal portion 126, a frontal portion 127, a mandibular portion 128, and temporal portions 129. The head band 111 may be configured to press the temporal portions 129 of the mask body 120 against the temporal areas of the patient's head. The head band 111 may be pivotally connected to the mask body 120 and may include a head band fastener 112 configured to tighten the head band 111 around the patient's head, so that the mask member 110 may be properly secured. In some embodiments, the mask member 110 may include additional straps/bands, that may be adjustable. For instance, an additional strap may extend from the glabellar portion 125 towards the occipital area of the patent's head and may connect with the headband 111 assisting in securing the glabellar portion 125 in place. In some embodiments, the fastening means may include a band 235 vertically extending from the glabellar portion 125, and that may connect to a central portion of the headband 111 located substantially in the middle of the headband 111. In some embodiments, the mask body 120 may include portions extending to temples of the patient, and/or areas extending to anterior auricular areas. In some embodiments, the mask body 120 may include an anterior portion extending above the glabellar portion and at last partially covering the forehead of a patient.

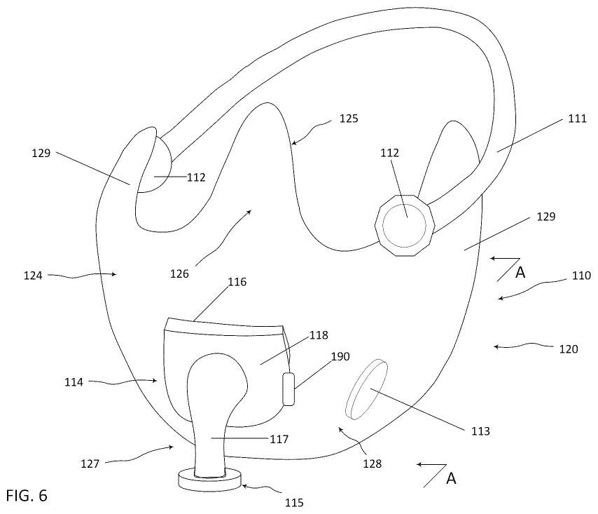

As best shown on FIGS. 5-6, the inlet portion 131 may be positioned in the frontal portion 127 of the mask body 120 and includes the inlet opening 122 and may further include a gas intake hatch 114 configured to couple with the gas conduit 180. The gas intake hatch 114 may include a conduit hub 115, configured to receive the gas conduit 180 and secure it to the inlet portion of the mask member. The gas intake hatch 114 encloses the inlet opening 122, and may be hingedly connected to the mask body 120 (for instance, by a hinge 116), so that the gas intake hatch 114 operable to open by swinging upwards, and to close forming a seal around the inlet opening 122. To ensure a substantially airtight seal, an upper (to-mask) portion of the gas intake hatch 114 and/or the inlet opening 122 of the mask body 120 may include (for instance, may be line up with) seal band 119 extending around at least a portion of their perimeters. The gas intake hatch 114 may be locked to the mask body 120 by the hatch release lock 190. In some embodiments, the hatch release lock 190 may be operatively connected to the controller 150 and configured to disengage based on the command received by the controller. The gas intake hatch 114 may include a chute 117 and a window 118; wherein the chute and/or the window may be made from transparent material; for instance, from transparent plastic but not limited thereto. The inspiration valve 191 may be located in the gas intake hatch 114; for instance, between the window 118 and the chute 117, as best shown on FIG. 9. In some alternative embodiments, the inspiration valve 191 may be positioned within the inlet opening 122. Moreover, at least one of the system monitoring means, such as FiO2 sensor, a humidity sensor, a fluid sensor gas flow sensor, and any combinations thereof may be positioned within the inlet portion of the mask body; for example, in at least one of the following: the gas intake hatch 114, the inspiration valve 191 and inlet opening 122.

In some embodiments, the mask body 120 may include system monitoring means comprising at least one aspiration detector 198, that may be connected to the mandibular portion 128 of the mask body 120, and may include a sensor configured to determine presence of fluid discharge (such as vomit, sputum, saliva and blood) in the cavity mask body 120, and wherein the aspiration detector 198 may be operatively connected to the controller 150, so that once the presence of the discharge is detected, the controller 150 may shut down the gas delivery assembly 140 (for instance the pump member 170), and cease ventilation of a patient. The system may further alert a care provider that the discharge is present within the mask body cavity by user alert means, so that the provider could promptly open the gas intake hatch 114 and apply suction. In some embodiments, the hatch release lock 190 may be configured to disengage based on the command received from the controller 150 when the discharge is detected and/or based on the input from the user. In some embodiments, a gas delivery unit, for instance the pump member 170, may be configured to directly couple to the inlet opening of the mask body 120.

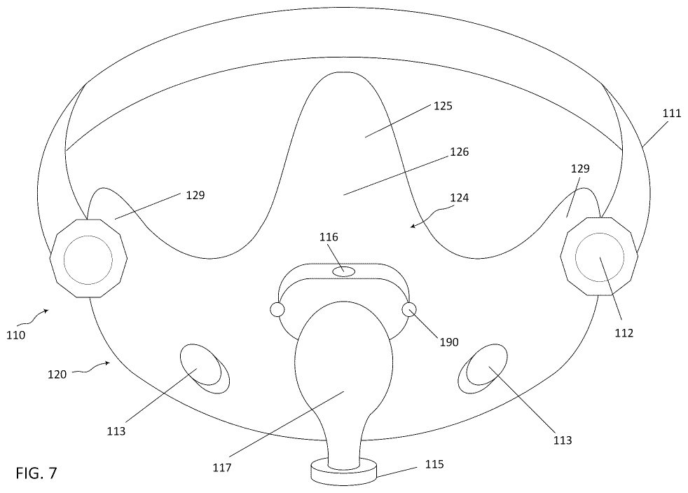

Referring to FIGS. 8-9. FIG. 8 is a cross-sectional view on the line A-A of the mask member of FIG. 6 and FIG. 9 is a back (into an inner cavity) view of the mask member illustrated in FIG. 7.

The one-way expiration valves 113 operatively connected to the controller and may be connected to the outlet portions 132, and in some embodiments may be imbedded in or connected to the outlet openings 121 of the mask body 120. The EVMA further includes system monitoring means comprising at least one pressure sensor 192 operatively associated with mask member 110 and configured to measure a pressure within the mask body 120. The pressure sensor 192 operatively connected with the mask body 120 and may be operatively connected to the controller 150. FIG. 8 shows the pressure sensor 192 as being included in the expiration valve 113. However, the location of the pressure sensor 192 is not limited, and it may be located elsewhere within the outlet portion 132 of the mask body 120; for instance, connected to the inner surface 123 of the mask body 120, connected to the expiration valve 113, or operatively connected to the inner surface 123 of mask body 120. The pressure sensor 191 may be operatively connected to the controller 150, so that when the gas pressure within the mask body exceeds the predetermined value, the controller 150 opens the expiration valves 113 and subsequently closes the valves 113 (or a single expiration valve 113, in some embodiments, wherein the mask member comprising a single outlet portion 132 including a single expiration valve 113) once pressure returns to a predetermined value. In some embodiments, the EVMA may include system monitoring means comprising at least one flow sensor operatively connected with mask body 120 and configured to measure the gas flow exiting airways of the patient, wherein the flow sensor may be operatively connected to the controller 150, and wherein the controller 150 configured to control the operation of the one-way expiration valve 113 based at least partially on a signal received from the flow sensor. The flow sensor may be configured to measure at least the direction of the flow (i.e. to determine that patient is exhaling) and duration of the flow; and wherein the controller 150 may control the expiration valve accordingly (keep it open for the duration of exhalation), and may use the data to estimate a lung volume of the patient (for instance, by multiplying a flow volume per time/duration of exhalation). The EVMA may include the pressure sensor 192 and/or the flow sensor, and wherein the expiration valve function may be controlled by the controller 150 based on signals from at least one of the sensors.

The mask member 110 includes the vitals sensor system 130 (exemplary configurations of which are shown on FIGS. 8-9) operatively connected to controller 150. The vitals sensor system 130 operatively connected to the mask member 110 and configured to measure signals associated with one or more physiological parameters of the patient. At least some physiological parameters data may be displayed by the user interface 160 or made available to care provider by any user alert means; for instance, displayed. The vitals sensor system 130 configured to measure the one or more physiological parameters comprising at least one of the parameters selected from the group consisting of: temperature, oxygen blood saturation (SpO2), heart rate, blood pressure, blood glucose level, partial pressure of carbon dioxide CO2 in the exhaled breath, positive end-expiratory pressure (PEEP), tidal volume, and combinations thereof. In some embodiments, the VSS 130 of the mask member may include at least one sensor/device configured to measure at least one cardiac function, such as stroke volume and/or cardiac output and/or arterial fibrillation detection, wherein the cardiac output is the volumetric flow rate of the heart's pumping output, and which may be the product of the heart rate, and the stroke volume, wherein the stroke volume is the volume of blood pumped from the left ventricle per beat. A variety of sensors known in the art for measuring the physiological parameters may be used. For example, and without limitation the heart rate and blood oxygen level (SpO2) monitoring may be performed by a photoplethysmography-based sensors and/or pulse oximeter; the partial pressure of carbon dioxide CO2 in the exhaled breath may be measured by a capnograph, the positive end-expiratory pressure (PEEP) may be a measured by a pressure and a flow monitor, the tidal volume may be measured by a flow sensor, the stroke volume may be measured by FloTrac sensor that uses arterial pressure waveform analysis and calculations, or other sensor relying on the arterial waveform analysis and/or doppler ultrasound flow sensor. The sensors of the VSS 130 are depicted schematically and not to scale, and their positions likewise may be approximate and exemplary. At least one of the following but not limited to: temperature, blood pressure, blood glucose level, heart rate, oxygen blood saturation (SpO2), cardiac output, may be measured at various facial/head areas of the subject. Thus, alternative arrangements of the sensors of VSS 130 are contemplated, operable and included in the scope of the current disclosure. For example, at least one the following physiological parameters that include but not limited to: temperature, blood pressure, blood glucose level, heart rate, oxygen blood saturation (SpO2), cardiac function, may be measured on at least one of the frontal, glabellar, nasal, temporal, mandibular or occipital areas according to locations of the facial/head blood vessels, and at least one of the corresponding sensors of the VSS may be positioned accordingly. The partial pressure of carbon dioxide CO₂ in the exhaled breath, positive end-expiratory pressure (PEEP), tidal volume sensors depiction as concentric circles is schematic and does not necessarily imply that one sensor may be positioned within another (as depicted on FIG. 8), but also does not exclude such option. The sensors of the VSS 130 may be imbedded between the inner and outer surfaces of mask body and/or mas body components, and/or connected to the inner and/or outer surfaces of the mask body and/or its components. The VSS 130 may be configured to transmit data to the controller 150 wirelessly or by conventional wired connection. The VSS 130 may further be configured to transmit data to additional user devices, such as cell phone, tablet, computer; and wherein the gas delivery assembly may be further configured to receive signals and be controlled from the user devices.

Furthermore, in some embodiments, at least one sensor of VSS 130 configured to measure at least one of the following physiological parameters that include but are not limited to: temperature, blood pressure, blood glucose level, heart rate, oxygen blood saturation (SpO2), cardiac function, may not be included in the mask body 120, and may be included in the fastening means of the mask member 110. In some embodiments, the fastening means includes a band in contact with and covering at least a portion of forehead/frontal area of the patient's face, and wherein the band may include at least one sensor of VSS 130 configured to measure at least one of the following physiological parameters that include but not limited to: temperature, blood pressure, blood glucose level, heart rate, oxygen blood saturation (SpO2), cardiac function. In some embodiments, the band may extend vertically above the glabellar portion of the mask member and may be fixedly connected or detachably coupled to the glabellar portion 125 of the mask body 120 and include a VSS pad in contact with at least a portion of the forehead/frontal area of the patient's face, and wherein the VSS pad may include at least one sensor of VSS 130 configured to measure at least one of the following physiological parameters that include but not limited to: temperature, blood pressure, blood glucose level, heart rate, oxygen blood saturation (SpO2), cardiac function. In some embodiments, sensors of VSS 130 configured to temperature, blood pressure, blood glucose level, heart rate, oxygen blood saturation (SpO2), cardiac function may be located in the fastening means, for instance in the VSS pad of the band.

Moreover, in some embodiments, at least one sensor configured to measure at least one the following physiological parameters that include but not limited to: temperature, blood pressure, blood glucose level, heart rate, oxygen blood saturation (SpO2), cardiac function may be not included in the VSS 130 of the mask member 110, and may not be included in the VSS 130 of the mask member 110, and may be included in the system 1000 as a separate unit operatively connected to the controller 150 and configured to measure at least one of the parameters at alternative locations of a patient's body.

The mask member may comprise any materials conventionally used for ventilation masks as known in the art. However, it is preferable that the inner surface 123 may be coated with antimicrobial substances or be comprised of a material having anti-microbial properties. Moreover, at least one antimicrobial material may be embedded between the inner and outer surfaces 123 and 124 of the mask body 120.

Furthermore, the mask body 120 may include embedded wires configured for connecting at last one sensor of the vitals sensor system 130 and may further connect the system control means 600 (for instance, the pressure sensor 192, but not limited thereto) with the controller 150; and wherein the controller 150 may be positioned on/in the gas delivery assembly 140, for instance in/on the pump member 170, but not limited thereto. In some implementations, the wires may continue along and/or be embedded within the gas conduit 180, as further described.

As shown, the mask member includes a vitals sensor system 130 that may include a tidal volume sensor 193, a capnograph 194, and a PEEP monitor 195, that may be included with the outlet openings 121, for instance in the expiration valves 113. At least one of the temporal portions 129 of the mask body 120 may include a temporal pulse sensor 196 configured to measure the heart rate behind the ears of the patient; for instance, from occipital and/or posterior auricular arteries. Additionally, or alternatively at least one pulse sensor may be located in the mandibular portion 128 of the mask body 120 and referred herein as mandibular pulse sensor 197 configured to measure a heart rate, for instance at external maxillary arteries. Moreover, the temporal portions 129 of the mask body 120 may include a section configured to extend to and at least partially cover the temples of a patient and at least one additional pulse sensor configured to measure the heart rate at the temporal arteries may be included. The controller 150 may be configured to average signals received from multiple pulse/heart rate sensors for more accurate readings.

The inner cavity of the mask body 120 may further include a nasal sensor pad 199 that may be located in the glabellar 125 and/or the nasal 126 portions of the mask body 120 and may have a glabellar area 204 and a nasal area 205 and configured to engage with the nasal and glabellar area of the patient's face. The nasal sensor pad 199 may host at least one sensor of the vitals sensor system 130, and may include at least one of the following: a thermometer 200, that may be located in on the glabellar area 204 of the pad 199; pulse oximeter 201, blood pressure monitors 202 and a non-invasive/bloodless blood glucose sensor 203 (that may include a microwave emitter/detector, optical sensor, PGG sensor, or combination thereof, but not limited thereto), at least one or each of which may be located in the nasal area 205 of the pad 199. However, the location of the sensors is not particularly limiting, and the pulse oximeter 201, the blood glucose monitor/sensor 202, the blood pressure sensor 203, and the thermometer 201 may be alternatively/additionally located elsewhere within the mask body 120 according to locations of facial/head blood vessels. For example, at least one of the sensors configured to measure at least one of physiological parameters comprising: temperature, heart rate, SpO2, blood pressure, blood glucose may be located in at least one of the following portions: the nasal portion 126, in the glabellar portion 125, mandibular portion 128, at least one temporal portions 129, and an anterior portion of the mask member 120 configured to extend from the glabellar portion 125 and configured to cover at least a portion of the forehead of the patient.

The inner surface/cavity of the mask body 120 may include lining and/or padding 206 that may be inflatable and/or extendable; and may be self-inflatable and/or extendable. The lining 206 may be configured to ensure a proper contact of at least one sensor of the vitals sensor system 130 with the area of the face/head wherein the sensor requires the proper contact to receive signal/collects the data. The mask body 120 may further include a lining bar 207, that may be positioned along the perimeter of the mask body and configured to restrict the lining, that may be inflatable, beyond the perimeter of mask body 120. The mask body 120 may include a liner that may be removable, and wherein the liner may be reusable or disposable.

In some embodiments, the mask body 120 may include an oxygen port (not shown) configured to receive an external oxygen source; for instance, an oxygen tank, chemical oxygen source; and wherein the port may further include means for controlled delivery of oxygen to the mask member 110, wherein the means may include a regulator, at least one sensor (that may include but not limited to a FiO2 sensor, pressure sensor etc.) that may be included in system monitoring means. The oxygen port may be used in situations wherein an immediate oxygenation of a patient is necessary. In some embodiments, the oxygen port may include additional sensors (that may include but not limited to a pressure sensor, a proximity sensor, a force sensor, an inductive sensor, a capacitive sensor, and any combinations thereof) that may be included in the system monitoring means and connected to the controller 150 and configured to detect that the oxygen source has been coupled to the oxygen port of the mask body 120; and wherein the controller 150 may be configured to automatically (and/or according to an input from a provider) shut off (or not turn on) the gas delivery assembly 140 (for instance, the pump member 170) when the oxygen source is coupled to the oxygen port. In some embodiments, wherein the oxygen port may be included in the mask body instead of one of the expiration valves 113, depicted on FIG. 7.

In some embodiments, the mask body 120 may include an auxiliary port (not shown) configured to receive at least one of the auxiliary modules 500; for instance, a nebulizer, but not limited thereto.

Though a particularly shaped mask member and mask body may be described herein in greater details and particular locations of the sensors of the vitals sensor system and the system monitoring means may be shown/mentioned and may be advantageous, alternatively shaped mask members and mask bodies and alternative location of the sensors of the vitals sensor system and system monitoring means will suffice and included in the scope of the current disclosure.

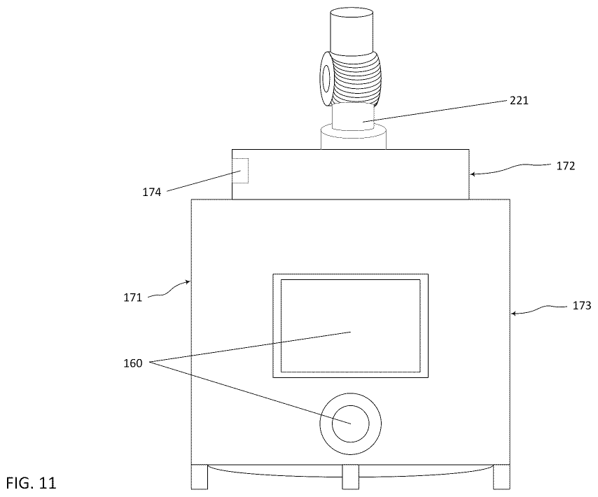

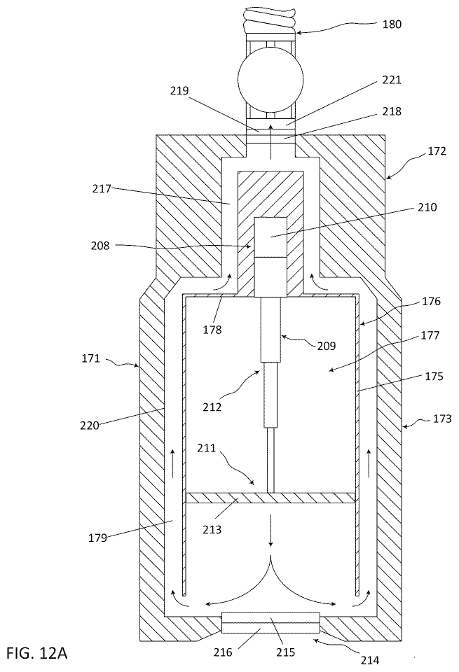

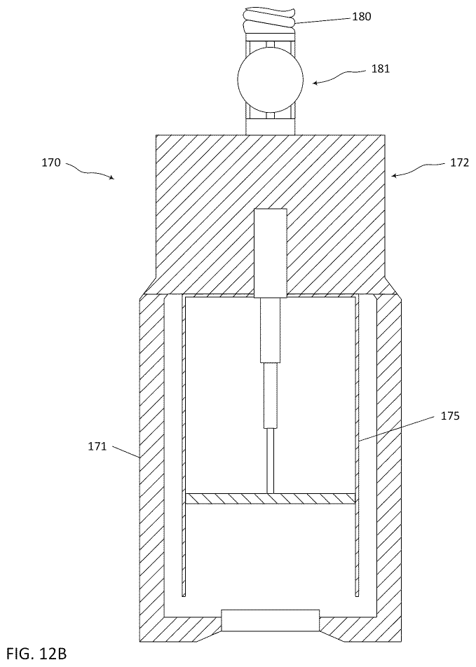

Referring now to FIGS. 10-12, an exemplary implementation of the gas delivery assembly 140 in accordance with an exemplary embodiment of the invention is now described in greater detail. The gas delivery assembly may include a pump member 170. FIG. 10 depicts a perspective view of the pump member 170; FIG. 11 depicts a front view; and FIG. 12 shows a cross-sectional view on the line B-B of the pump member of FIG. 10 and FIG. 11. As shown, the pump member 170 may include an outer jacket 171, wherein the jacket 171 may include an upper section 172 and a lower section 173. The jacket 171 may include the controller 150, (not shown) and/or a user interface 160. In some embodiments, the upper section 172 of the jacket 171 may include a charging port 174 for a battery that is configured to deliver power to moving components of the pump member 170. The battery may be housed within/on the jacket 171 and, for instance, may be housed within/on the upper section 172. The location of the battery is not limiting, and alternative exemplary locations for the battery are possible and included in the current disclosure. Furthermore, the battery may be removable, and/or rechargeable. The pump member 170 may include an open-ended, or at least partially open-ended drum 175 positioned within a jacket 171, and wherein the drum 175 may have an outer surface 176, a drum cavity 177, and a drum roof 178. The pump member 170 may include a piston assembly 209 that may include a piston 211 and piston driving means 211, the means operatively connected to the controller 150, and wherein the means may include a piston actuator 210. The piston 211 may include a leg 212 and a plunger 213; and wherein the leg 212 may be configured as a telescopic leg (i.e., being length-adjustable, such as extendable/retractable and/or foldable) operatively connected to the piston actuator 210 and extending between the actuator 210 and the plunger 213; and wherein the actuator 210 may drive the leg 212 to extend and contract. A piston hub 208 configured to house/contain at least the piston actuator 210; and may be further configured to house/receive at least a portion of the piston leg 212. The drum roof 178 may include a piston hub 208. The piston leg 212 may be configured to expand and contract as operated by the piston actuator 210, so that the plunger 211 may move up and down within the drum cavity 177. The jacket 171 may include an inlet port 214 that may be equipped with a one-way lower pump valve 215, and an outlet port 218 that may be equipped with a one-way upper pump valve 219; and wherein at least one of the valves configured as a one-way valve/membrane/diaphragm. The inlet port 214 may further include a filter 216 (for instance, but not limited to a HEPA filter), configured to purify the gas that enters the pump member 170 form atmosphere and/or from auxiliary module(s) 500. The filter 216 may include a filter cap configured to cover the filter 216 when the system 1000 is not in immediate use. The system 1000 may be further configured to alert the user/provider when a flow through the filter 216 becomes insufficient and the filter change is required. The piston actuator 210 may be operatively connected to the controller 150 (not shown).

When the leg 212 is being contracted and the plunger 213 moves upwards in the direction of the drum roof 178, the piston 211 is being retracted and creates a negative pressure/vacuum in the drum cavity 177 and the gas enters the cavity 177 from the atmosphere (and/or at least one of the auxiliary modules) through the open one-way lower pump valve 215, while the upper pump valve 219 remains closed. Moreover, the expiration valves 113 of the mask member 110 (best shown on FIGS. 2-7) may be configured to be open when the piston 211 is being retracted and an exhalation is detected by the pressure sensors 192; and if no exhalation is detected (e.g., a predetermined value hasn't be reached as determined by a pressure sensors 192), the expiration valves 113 may remain closed regardless of the retracting action of the piston 211. In some alternative embodiments, the expiration valve 113 may remain open at all times when the piston 211 is retracting.

When the leg 212 is being extended and the plunger 213 moves downwardly, the lower pump valve 215 remains closed, and the gas flow is directed by the plunder 211 from the drum cavity 177 into a gas duct 179 and further up to gas channel(s) 217 wherein the gas flow exits the pump member through the opened upper pump valve 219 and into the gas conduit 180, from which it subsequently enters the mask member through the inspiration valve 191. Moreover, the expiration valves 113 of the mask member 110 (best shown on FIGS. 2-7) may be configured to be closed when the piston 211 is being extended.

The lower pump valve 215, the upper pump valve 219, the inspiration valve 191, and the expiration valve(s) 113, each may be configured as any or the following: one-way valves, membrane or diaphragm valves; and open and closed based on a command from the controller 150 according to pre-determined algorithm associated with the piston motion and/or controlled locally by operatively connected pressure sensor(s), and/or mechanically as pre-determined by particular structure of the valve/membrane/diaphragm.

As depicted on FIGS. 12A-B, in some embodiments, the gas duct 179 may include a hollow space defined by an inner wall 220 the jacket and the outer surface 176 of the drum 175, and the gas channels 217, that may comprise a pair of tubular passages (such as tubes, but not limited thereto) extending between the gas duct 179 and the outlet port 218.

In some alternative embodiments, the gas duct may include at least one tube, but preferably a pair of tubes configured to convey the gas from the drum 175 to the gas channels 217 as directed by the piston 211. Moreover, in yet some other alternative embodiments, a single gas channel 217 is contemplated, that may include a hollow space defined by a jacket 171 and a piston hub 208, and wherein the gas duct may include a single channel defined by the jacket inner wall 220 and the outer surface 176 of the drum 175 (as the gas duct 179 depicted on FIG. 12), or alternatively include at least one passage that may be tubular.

The number of gas ducts and gas channels is not particularly limiting, and though a pair of gas channels 217 and a single gas duct 179 is depicted on the FIG. 12, the pump member 170 may include at least one gas duct 179 and/or at least one gas channel 217.

The pump member 170, and for instance the inner port 214 of the pump member 170 may be connected to or configured to detachably receive at least one auxiliary module 500, that may include, but not limited to at least one device selected from: an oxygen source (that may be an oxygen tank or the source configured to generating oxygen by a chemical reaction) configured to supply (and may be further configured to regulate an amount/concentration) of oxygen in the gas flow directed to the patient by the pump member 170, a humidifier operable to increase moisture/water content in the gas flow directed to the patient by the pump member 170, a dehumidifier operable to reduce the moisture content directed to the patient by the pump member 170, a nebulizer operable to deliver a medicine/treatment into the gas flow directed to the patient by the pump member 170. For instance, the input from the SpO2/oximeter sensor 201 may be processed by the controller 150, and wherein the data are indicative that the patient blood oxygen saturation is lower than pre-determined value, the controller 150 may send the command to the auxiliary oxygen source to increase the concentration of the oxygen (FiO2) in the gas flow directed to the mask body 120 by a pump member 170. The pump member 170 and/or gas conduit 180 and/or the mask member 110 may include at least one sensor configured to determine the FiO2 in the gas flow. In some embodiments, the pump member 170 may include a pump terminal 221 configured to couple with the gas conduit 180 and/or the conduit management assembly 181. The system may include connecting means for coupling at least one of the auxiliary modules and/or supporting modules to the gas delivery assembly. The pump member may include the connecting means for connecting at least one of the auxiliary modules and/or supporting modules thereto.

In some embodiments the volume of the drum 175 may be about 5-11 liters; preferably about 6-10 liters; and most preferably about 7-9 liters. In some embodiments, the volume of the drum 175 may be about 7.5-8.5 liters, and preferably about 8 liters. In some embodiments, the height of the drum 175 may be 5-10 inches, preferably about 6-9 inches, and most preferably about 8 inches. In some embodiments, the diameter of the drum may be about 3-10 inches, preferably about 4-7 inches, and most preferably about 5 inches. The jacket 171 may be made of wholly or partially from at least one of the following: metal, metal alloy, plastic, polymer composite or any combination thereof.

Though a particular pump member is disclosed and described in greater details herein, other pumps, and particularly pumps operable to deliver an intermittent and controlled gas flow to the mask member can and will suffice and included in the scope of the current disclosure.

Referring now to FIG. 13 showing an exemplary embodiment of the gas conduit 180. The conduit 180 may include a tube 183 configured to extend between the mask body 120 and the pump member 170. The gas conduit may include a housing 184 configured to extend around the tube 183. The tube 183 and the housing 184 may be configured to collapse and extend in an accordion-like or spring-like manner. For instance, the tube may include a coiled spine 186 that may be configured to support the tube 183 and enables it to extend and collapse in pre-determined accordion or spring-like manner. Alternatively, the spine may be not included, and the tube may be fabricated in a shape that enables the tube to collapse and extend in an accordion or spring-like manner. The tube may be made of plastic, and preferably made of the plastic having anti-microbial properties. The gas conduit 180 and/or tube 183 may be corrugated is an alternative manner than depicted.

The housing 184 may include at least one bore 185, that may be configured as a spiral hollow passage that may coil around the tube 183 and may host the wires extending between the vitals sensor system 130 and/or system control means to the controller 150; wherein the controller 150 may be positioned within/on the pump member 170.

The gas conduit 180 may include an upper clip 188 configured to couple to the mask body 120, for instance to the conduit hub 115; and the lower clip 189 configured to couple to the pump terminal 221 and/or conduit management assembly 181.

Though a particular gas conduit is disclosed and described in greater details herein, other conduits operable to conduct gas flow to the mask member can and will suffice and are included in the scope of the current disclosure.

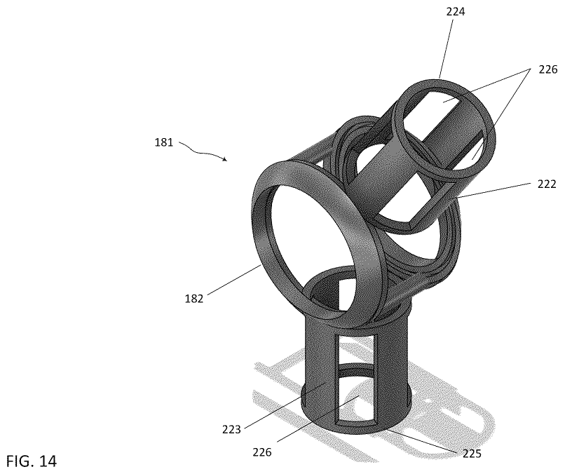

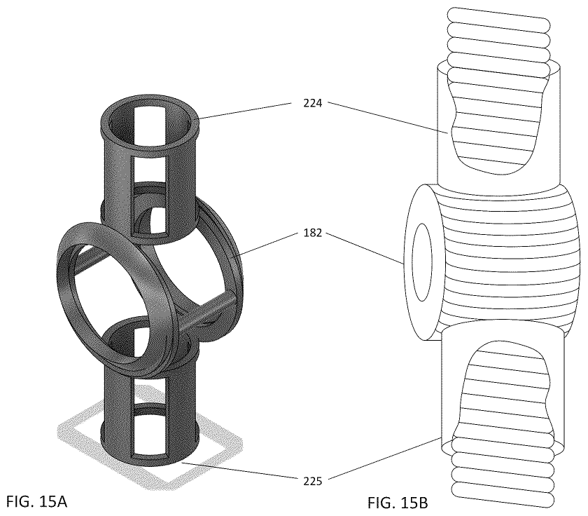

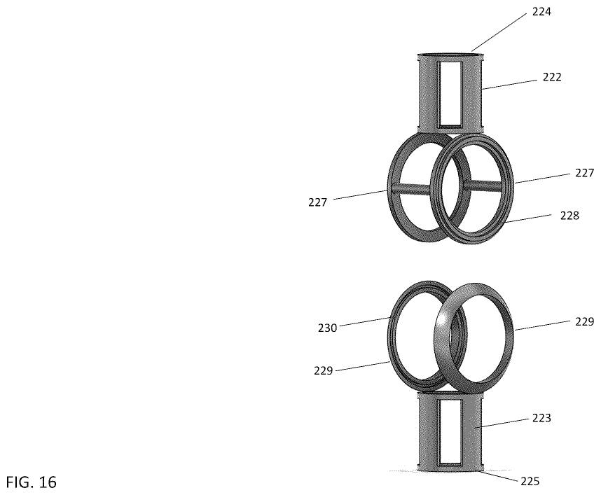

Referring now to FIG. 14-16 showing an exemplary embodiment of the conduit management assembly 181. FIG. 14 and FIG. 15 show perspective views of the conduit receiving assembly 181, wherein the assembly is shown in bent and straightened positions respectively. FIG. 16 shows an exploded view of assembly 181. As shown, the conduit management assembly 181 may include a knee 182 positioned between an upper guide 222 and a lower guide 223. The upper guide 222 may include an upper spout 224 that may define a circular orifice. The upper spout 224 being fixedly connected to the conduit hub 115 or may be configured to detachably couple to the conduit hub 115, for instance by fastening means. The lower guide 223 may include a lower spout 225 that may define a circular orifice. The lower spout 225 be fixedly connected to the pump terminal 221 or may be configured to detachably couple to the pump terminal 221, for instance by fastening means. The fastening means may include at least one of the following, but not limited to clips, clasps, pins, latches, locks, grooves, apertures, and magnets.

In some embodiments the upper spout 224 may be configured to detachably couple to the conduit hub 115 and the lower spout 225 may be configured to detachably couple to the pump terminal 221. In some alternative embodiments, the upper spout 224 may be configured to detachably couple to the conduit hub 115 and the lower spout 225 may be configured to be fixedly connected to the pump terminal 221.

The conduit management assembly 181 may be configured to receive the gas conduit 180, so the gas conduit may enter through an upper spout 224, pass through the upper guide 222, pass through the knee 182, pass through the lower guide 223, and may exit through the lower spout 225. It should be noted that relative terms “upper”, “lower” and such are used herein and through the entirety of the disclosure are used solely for and do not limit orientation of the described object. For instance, the conduit management assembly 181, may be oriented otherwise, and either the upper spout 224 or the lower spout 225 may be configured to couple to the conduit hub 115 and/or the pump terminal 221.

The conduit management assembly 181 may be configured to receive a portion of the length and/or an entirety of the length substantially contracted gas conduit 180. The upper spout 224 and the lower spout 225 may be sized, so the gas conduit 180 may pass through. However, it is contemplated and included in the scope of the disclosure that the conduit management assembly 181 may be configured to receive a portion of the gas conduit 180 and/or selectively receive a portion of the gas conduit 180, so that the length of the gas conduit 180 may be adjustable.

The gas conduit management assembly 181 may include a gripping clasp (not shown) that may be included in the upper guide 222 (for instance in the upper spout 224, but not limited thereto), that configured to adjust the length of the gas conduit 180 by releasably gripping it's outer surface, so that the gas conduit 180 may be contracted into the management assembly 181 until desired length (that may be necessary to span between the pump member and the mask member) is achieved and secured at this length by the gripping clasp; and may provide a great convenience to a care provider by adjusting the length of the conduit according to a particular situation/setting, and may further improve safety for both patient and the provider by preventing entangling of the gas conduit on itself or with other objects, that may contribute to trauma and cause conduit damage.

The ability of the conduit management assembly 181 to enclose an entirety of the gas conduit 180 (as shown on FIG. 4), likewise enhances convenience, safety and provide a storage enclosure for the conduit 180 when EVMA 100/system 1000 are not in an immediate use, as in storage or transport.

The upper guide 222 and/or the lower guide 223 may define at least one window 226, extending through, so that the gas conduit 180 may be visible, and may manually be popped out if accidentally gets stuck within.

The knee 182 may enable bending of the assembly 181, without pinching the gas conduit 180 that passes through. As shown on FIG. 16, the knee 182 may comprise a pair of set apart inner hoops 227 each of which may include extrusion 228, and a pair of set apart outer hoops 229, each of which may include a divot 230; and wherein the extrusions 228 of the inner hoops 227 may be configured to couple with and rotate within the divots 230 of the outer hoops 229, so that the conduit receiving assembly 181 may be bent and straightened around the knee 182.

Though a particular conduit management assembly is disclosed and described in greater details herein, other structures operable in a similar manner can and will suffice and included in the scope of the current disclosure.

The system comprising EVMA may include at least one handle that may include a non-slip grip, such as a rubberized grip configured to enable secure holding and transportation of the EVMA apparatus 100/system 1000. The grip may be positioned on the gas blowing assembly, for instance on the gas pump 170, but not limited thereto.

In some embodiments, an energy source to power the EVMA and/or the system may include a battery but not limited thereto. The energy source, such as battery that may be configured to couple to the gas delivery assembly 140, for instance to the pump member 170. The gas delivery assembly 140; for instance, the pump member 170, may include a compartment or terminal easily accessible from the outer surface of the gas delivery assembly/pump member, therein the battery may be easily inserted to and removed from the compartment/terminal for easy and fast replacement. The battery may be configured to couple magnetically to the gas delivery assembly/pump member.

In other embodiments the battery may be integrated into the EVMA/system and may be rechargeable.

The battery may be alternatively housed in/on auxiliary module(s) 500 and configured to power the system 1000. In some embodiments at least one auxiliary module 500 may include its own power source.

The EVMA/system may include a mechanism that prevents removal of the battery or a module housing the batter while the EVMA/system is in use (switched “on”). The battery module/battery may include at least one, but preferably two or more buttons that may need to be pressed simultaneously eject or remove the battery, preventing accidentally battery ejection while EVMA/system is in use (“on”). If battery is attempted to be ejected while EVMA/System is operation, the battery/battery module will not eject unless manual confirmation from provider is received through the user interface. The user interface may display information relating to the battery, such as percentage of the charge remaining and estimated remaining operation time, as well as give alerts if battery is low visually or audibly.

In some embodiments at least of the auxiliary module may require an adaptor to couple to the EVMA.

In some embodiments the filter 216 may be removable and may need to be removed before the auxiliary module(s) may be coupled thereto. For instance, when coupling the nebulizer auxiliary module and/or humidifier.

The EVMA 100 may include a tracheotomy ventilation accessory; that may include, but not limited to a tracheotomy tube. The tracheotomy ventilation accessory may be configured to couple to the mask body 120. The EVMA may include a suction accessory configured to suction/clear patient airways and/or interion cavity of mask member 120. The tracheotomy accessory and/or the suction accessory may be removable, may be fixedly at least partially fixedly connected to the mask member, or may be included as a kit and be stored elsewhere within the system when not in use.

Different features, variations and multiple different embodiments of the EVMA as whole and its parts and modules have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiment or enumerated variations. Many modifications, variations and other embodiments will come to mind of those skilled in the art, and which are intended to be and are in fact covered by this disclosure. It is intended that the scope of this disclosure should be determined by a proper legal interpretation and construction of the disclosure, including equivalents, as understood by those of skill in the art relying upon the complete disclosure present at the time of filing. It is intended that the claims be interpreted to embrace all such alternative forms, modifications, variations, embodiments, equivalents, and modification where applicable.

### Utility

The utility of the system 1000 comprising EVMA 100 includes, but is not limited to the following. The system 1000 is an elegant and robust solution that substantially eliminates the need for critical care providers to be engaged in measuring, evaluating and tracking the multitude of physiological parameters of the patient by applying multiple devices to multiple body areas, while maybe as well providing needed ventilation. The system 1000 may be configured and operable not only to administer ventilation to a patient, but also to provide immediate assessment and monitoring of the patient and may be configured as a portable and compact apparatus that requires a single care provider to efficiently and accurately operate. The system 1000 may be configured to measure a multitude of physiological parameters reducing the need for multitude of auxiliary devices or attachments for measuring physiological parameters and may be configured as all-in-one system operable to administer ventilation to a patient and to provide critical assessment and monitoring of the patient by measuring a multitude of physiological parameters while requiring only a single area of body contact. The system 1000 may be configured to operate in a feedback-controlled mode and adjust ventilation parameters based on at least partially on the measured physiological parameters, wherein such adjustment may be delivered automatically and/or manually.

The system 1000 offers an unsurpassed convenience, particularly to first responders, such as EMTs and paramedics, by enabling a single care provider to accomplish most of the initial assessment, ventilating, and monitoring tasks in a fast, efficient, accurate and substantially hands-free manner, and thus allowing the critical care provider(s) to focus on and administer additional lifesaving treatments. Moreover, the invention reduces initial assessment time and provides a fuller clinical picture by simultaneously measuring and making available to care provider the multitude of patient's physiological parameters, thus greatly improving quality of care. The system 1000 and its individual components may be particularly advantageous in critical and emergency care settings. However, the system 1000 and its individual components may be used in stationary setting such hospitals and long-term care facilities. The system 1000 may be used by the patient to self-administer ventilation when practical.

The system 1000 may be configured to deliver intermittent breaths to a patient and may be configured to operate and switch between the CPR and non-rebreather sub-modes. The system may also be configured to provide continuous ventilation to the patient. The system may include advantageous auxiliary and supporting modules.

Insofar as the description above and accompanying drawings disclose any additional subject matter that is not within the scope of claims below, the inventions are not dedicated to the public and the right to file one or more applications to claims such additional inventions is reserved.

What is claimed is:

1. An emergency ventilating and monitoring system, the system comprising an emergency ventilating and monitoring apparatus (EVMA), the apparatus comprising:
a. a mask member comprising a mask body and a vitals sensor system;
wherein the mask body having an inner surface configured for engagement with at least partial facial area of a subject, and an opposed outer surface; wherein the mask body includes an inlet portion and an outlet portion; the inlet portion comprising an inlet opening defined by the mask body and extending between the inner and the outer surfaces of the mask body, and the outlet portion comprising an outlet opening defined by the mask body and extending between the inner and the outer surfaces of the mask body; and
wherein the vitals sensor system operatively connected to the mask member and comprising one or more sensors configured to measure signals associated with one or more physiological parameters of the subject;
b. a gas delivery assembly operatively connected to the inlet portion of the mask body and configured to generate and direct a gas flow with defined ventilation parameters to the inlet portion of the mask body;
c. a controller operatively connected to the gas delivery assembly and the vitals sensor system of the mask member and configured to receive the signals associated with a plurality of physiological parameters of the patient from the vitals sensor system of the mask member, process the signals, and control the ventilation parameters of the gas delivery assembly based at least partially on the signals associated with at least one of the physiological parameters received from the vitals sensor system of the mask member;
d. a user interface operatively connected to the controller, wherein the user interface comprising user alert means and user control means; wherein the user control means configured to receive an input from the user to the controller, and the user alert means configured to provide an output to the user from the controller; and wherein the controller further configured to control the ventilation parameters of the gas delivery assembly based at least partially on the input from the user;
e. a one-way inspiration valve operatively connected to the gas delivery assembly and the inlet portion of the mask body and configured to allow the gas flow from the gas delivery assembly to the mask member and to prevent backflow;
f. at least one one-way expiration valve operatively connected to the outlet portion of the mask body and configured to allow the gas flow from the mask body to the atmosphere; and wherein the expiration valve operatively connected to the controller and configured to open and close based at least partially on a command received from the controller;
g. at least one system monitoring means operatively connected to the controller, and to at least one of the following: the mask member, the gas delivery assembly; and wherein the means configured to at least send a signal to the controller; and wherein the controller further configured control the ventilation parameters of the gas delivery assembly and operation of the one-way expiration valve based at least partially on a signal received from at least one of the system monitoring means.
and wherein the system configured to operate in a feedback controlled mode;
and wherein the system configured to be connected to a source of power.

2. A system as in claim 1, wherein the one or more physiological parameters comprising at least one physiological parameter selected from the group consisting of: temperature, blood oxygen saturation (SpO2), heart rate, blood pressure, blood glucose level, partial pressure of carbon dioxide in the exhaled breath, positive end-expiratory pressure (PEEP), tidal volume, cardiac function, and combinations thereof.

3. A system as in claim 1, further comprising one or more auxiliary modules operatively connected to the controller and the gas delivery assembly, wherein the one or more auxiliary modules comprising at least one auxiliary module selected from the group consisting of a humidifier, a dehumidifier, an oxygen source, an air/oxygen mixer, a nebulizer, an air heater, and combinations thereof.

4. A system as in claim 1, further comprising one or more supporting modules, wherein the one or more supporting modules comprising at least one supporting module selected from the group consisting of an IV fluids warmer, suction pump, body fluids receptacle, a storage unit, a refrigeration unit, and combinations thereof.

5. A system as in claim 1, wherein the inspiration valve operatively connected to the controller and configured to open and close based at least partially on a command received from the controller.

6. A system as in claim 1, wherein the mask member may further comprising fastening means configured to secure the mask body on the head of the subject, and wherein at least one of the sensors of the vitals sensor system operatively connected to at least one of the fastening means.

7. A system as in claim 1, wherein the mask body further comprising at least one of the following: an oxygen port, an auxiliary port.

8. A system as in claim 1, wherein the gas delivery assembly comprising a gas delivery unit, wherein the gas delivery unit comprising at least one device selected from the group consisting of: a pump, a blower, a compressible bag, and combinations thereof, and wherein the gas delivery unit having an inlet port and an outlet port.

9. A system as in claim 8, wherein the gas delivery assembly further comprising a gas conduit configured to direct a gas flow from the gas delivery unit to the inlet portion of the mask body and extending between the gas delivery unit and the inlet portion of the mask body, the gas conduit having a first end and an opposing second end, wherein the first end configured to couple to the inlet portion of the mask body and the second end configured to couple to the outlet port of the gas delivery unit.

10. A system as in claim 9, wherein the gas delivery assembly further comprising a conduit management assembly configured to at least partially receive at least a portion of the gas conduit.

11. A system as in claim 1, wherein the gas delivery assembly comprising a pump member having an inlet port and an outlet port, wherein the gas delivery assembly further comprising a gas conduit configured to direct a gas flow from the pump member to the inlet portion of the mask body and extending between the pump member and the inlet portion of the mask body, the gas conduit having a first end and an opposing second end, wherein the first end configured to couple to the inlet portion of the mask body and the second end configured to couple to the outlet port of the pump member.

12. A system as in claim 11, wherein the gas delivery assembly further comprising a conduit management assembly configured to at least partially receive at least a portion of the gas conduit.

13. A system as in claim 1, wherein the gas delivery assembly comprising a pump member, wherein the pump member comprising; a jacket, the jacket having an upper portion and a lower portion, wherein the lower portion including an inlet port and the upper portion including an outlet port;
the jacket having an inner wall and an outer wall;
at least partially open-ended drum positioned within the jacket, the drum having an outer surface and a drum cavity and a drum roof;
a gas duct defined by the outer surface of the drum and the inner wall of the jacket; a piston assembly comprising a piston and piston driving means, wherein the piston having a led and a plunger, the leg having a first end and an opposing second end, and wherein the fist end of the leg connected to the plunger and the second end of the leg connected to the piston driving means, and wherein the piston driving means connected to the drum roof and operatively connected to the controller;
a gas channel extending between the gas duct and the outlet port.

14. A system as in claim 13, wherein the gas delivery assembly further comprising a gas conduit configured to direct a gas flow from the pump member to the inlet portion of the mask body and extending between the pump member and the inlet portion of the mask body, the gas conduit having a first end and an opposing second end, wherein the first end configured to couple to the inlet portion of the mask body and the second end configured to couple to the outlet port of the pump member.

15. An emergency ventilating and monitoring system, the system comprising an emergency ventilating and monitoring apparatus (EVMA), the apparatus comprising:
a. a mask member comprising a mask body and a vitals sensor system;
wherein the mask body having an inner surface configured for engagement with at least partial facial area of a subject, and an opposed outer surface; wherein the mask body includes an inlet portion and an outlet portion; the inlet portion comprising an inlet opening defined by the mask body and extending between the inner and the outer surfaces of the mask body, and the outlet portion comprising an outlet opening defined by the mask body and extending between the inner and the outer surfaces of the mask body; and
wherein the vitals sensor system operatively connected to the mask member and comprising one or more sensors configured to measure signals associated with physiological parameters of the subject, wherein physiological parameters comprising: temperature, blood oxygen saturation (SpO2), heart rate, blood pressure, partial pressure of carbon dioxide in the exhaled breath, positive end-expiratory pressure (PEEP), and tidal volume;
b. a gas delivery assembly operatively connected to the inlet portion of the mask body and configured to generate and direct a gas flow with defined ventilation parameters to the inlet portion of the mask body;
c. a controller operatively connected to the gas delivery assembly and the vitals sensor system of the mask member and configured to receive the signals associated with a plurality of physiological parameters of the patient from the vitals sensor system of the mask member, process the signals, and control the ventilation parameters of the gas delivery assembly based at least partially on the signals associated with at least one of the physiological parameters received from the vitals sensor system of the mask member;
d. a user interface operatively connected to the controller, wherein the user interface comprising user alert means and user control means; wherein the user control means configured to receive an input from the user to the controller, and the user alert means configured to provide an output to the user from the controller; and wherein the controller further configured to control the ventilation parameters of the gas delivery assembly based at least partially on the input from the user;
e. a one-way inspiration valve operatively connected to the gas delivery assembly and the inlet portion of the mask body and configured to allow the gas flow from the gas delivery assembly to the mask member and to prevent backflow;
f. at least one one-way expiration valve operatively connected to the outlet portion of the mask body and configured to allow the gas flow from the mask body to the atmosphere; and wherein the expiration valve operatively connected to the controller and configured to open and close based at least partially on a command received from the controller;
g. at least one system monitoring means operatively connected to the controller, and to at least one of the following: the mask member, the gas delivery assembly; and
wherein the means configured to at least send a signal to the controller; and wherein the controller further configured control the ventilation parameters of the gas delivery assembly and operation of the one-way expiration valve based at least partially on a signal received from at least one of the system monitoring means.
and wherein the system configured to operate in a feedback controlled mode;
and wherein the system configured to be connected to a source of power.

16. A system as in claim 15, wherein physiological parameters further comprising at least one of the following: blood glucose level, cardiac function, and combinations thereof.

17. A system as in claim 15, wherein the mask body further comprising at least one of the following: an oxygen port, an auxiliary port, and combination thereof.

18. A system as in claim 15, wherein the gas delivery assembly comprising a gas delivery unit, wherein the gas delivery unit comprising at least one device selected from the group consisting of: a pump, a blower, a compressible bag, and combinations thereof, wherein the gas delivery unit having an inlet port and an outlet port, wherein the gas delivery assembly further comprising a gas conduit configured to direct a gas flow from the gas delivery unit to the inlet portion of the mask body and extending between the gas delivery unit and the inlet portion of the mask body, the gas conduit having a first end and an opposing second end, wherein the first end configured to couple to the inlet portion of the mask body and the second end configured to couple to the outlet port of the gas delivery unit.

19. A system as in claim 15, wherein the gas delivery assembly further comprising a conduit management assembly configured to at least partially receive at least a portion of the gas conduit.

20. A system as in claim 15, wherein the gas delivery assembly comprising a pump member having an inlet port and an outlet port, wherein the gas delivery assembly further comprising a gas conduit configured to direct a gas flow from the pump member to the inlet portion of the mask body and extending between the pump member and the inlet portion of the mask body, the gas conduit having a first end and an opposing second end, wherein the first end configured to couple to the inlet portion of the mask body and the second end configured to couple to the outlet port of the pump member.

21. A system as in claim 15, wherein the gas delivery assembly comprising a pump member, wherein the pump member comprising; a jacket, the jacket having an upper portion and a lower portion, wherein the lower portion including an inlet port and the upper portion including an outlet port; the jacket having an inner wall and an outer wall;
at least partially open-ended drum positioned within the jacket, the drum having an outer surface and a drum cavity and a drum roof;
a gas duct defined by the outer surface of the drum and the inner wall of the jacket;
a piston assembly comprising a piston and piston driving means, wherein the piston having a led and a plunger, the leg having a first end and an opposing second end, and wherein the fist end of the leg connected to the plunger and the second end of the leg connected to the piston driving means, and wherein the piston driving means connected to the drum roof and operatively connected to the controller;
a gas channel extending between the gas duct and the outlet port.

22. A system as in claim 21, wherein the gas delivery assembly further comprising a gas conduit configured to direct a gas flow from the pump member to the inlet portion of the mask body and extending between the pump member and the inlet portion of the mask body, the gas conduit having a first end and an opposing second end, wherein the first end configured to couple to the inlet portion of the mask body and the second end configured to couple to the outlet port of the pump member.

23. A system as in claim 22, wherein the gas delivery assembly further comprising a conduit management assembly configured to at least partially receive at least a portion of the gas conduit.

24. A system as in claim 15, wherein the mask body further comprising at least one of the following: an oxygen port, an auxiliary port, and combination thereof.

25. A system as in claim 15, wherein the mask member may further comprising fastening means configured to secure the mask body on the head of the subject, wherein at least one of the sensors of the vitals sensor system operatively connected to at least one of the fastening means.

26. A system as in claim 15, wherein the inspiration valve operatively connected to the controller and configured to open and close based at least partially on a command received from the controller.

27. A system as in claim 15, further comprising at least one of the following: an auxiliary module, supporting module, and combinations thereof, and wherein at least one of the auxiliary modules connected to the controller.
